(19) United States
(12) Patent Application Publication
(10) Pub. No.: US 2011/0287010 A1
(43) Pub. Date: Nov. 24, 2011

(54) Diagnostic methods and markers

(75) Inventors: Stephen John Assinder, New South Wales (AU); Jo-Ann Stanton, Dunedin (NZ)

(73) Assignee: Otago Innovation Limited, Dunedin (NZ)

(21) Appl. No.: 12/308,008

(22) PCT Filed: Jun. 7, 2007
(86) PCT No.: PCT/NZ2007/000142

§ 371 Date: Aug. 20, 2009

Related U.S. Application Data
(60) Provisional application No. 60/811,407, filed on Jun. 7, 2006.

(51) Int. Cl. A61K 39/395; C12N 15/00; C12N 5/06; C07K 14/435; C07K 16/18; C12P 21/02; C40B 40/08; C12Q 1/68; G01N 33/566; C07H 21/00; A61K 31/7088; A61K 31/7105; A61K 39/00; A61K 38/16; A01K 67/027; C12N 15/11

(52) U.S. Cl. 424/139.1; 536/23.5; 435/320.1; 435/325; 530/350; 530/387.9; 530/387.3; 530/391.3; 435/69.1; 506/17; 436/501; 536/24.5; 514/44 A; 424/277.1; 514/44 R; 800/18

(57) ABSTRACT

The present invention relates to methods of detecting, monitoring and treating prostate cancer (PRC) OR prostatic intraepithelial neoplasia (PIN) or a predisposition to same. Provided for use in the methods is a novel cancer marker, PSPU43, as well as bioassays and kits.

### FIELD OF THE INVENTION

This invention relates to methods of detecting, diagnosing, monitoring and treating prostate cancer (PRC), prostatic intraepithelial neoplasia (PIN) or a predisposition to same; and to markers useful in such methods.

### BACKGROUND OF THE INVENTION

Prostate cancer is the most commonly diagnosed cancer in European and North American men. In those regions prostate cancer is second only to lung cancer as a cause of death in men (Frankel et al. 2003). The disease is also on the increase in other parts of the world such as Japan, and may reflect an adoption of Western diets in Eastern Countries.

Prostate cancer is a disease of the aging male. Forty percent of men aged 60 years have localised prostate tumours, and more than 75 percent of men aged 85 years and older have prostate cancer. The cancer is a latent disease often present without other signs of disease, and can take up to 10 years from diagnosis to death. The disease's usual progression is from a well defined mass within the prostate to a breakdown and invasion of the lateral margins of the prostate, followed by metastasis to regional lymph nodes, and/or metastasis to bone marrow.

Prostatic intraepithelial neoplasia (PIN) is a specific type of lesion that is believed to be a precursor to prostate cancer (McNeal and Bostwick, 1986). If diagnosed early, patients are currently treated by androgen ablation therapy. Ablation therapy has undesirable side effects such as loss of libido and potency. As the disease develops it becomes androgen independent. At that stage surgery (radical prostatectomy) is the main option employed. The patient's life may be saved but common outcomes of surgery are incontinence, erectile dysfunction and urinary leakage.

It remains unclear why prostate cancer develops and what determines its progression. Moreover, tests for prostate cancer are limited primarily to physical examinations, needle biopsy and bone scan. Currently, a raised level of circulating prostate specific antigen (PSA) is most commonly used to predict the presence of prostate cancer. This is the only common non-invasive screen for prostate cancer.

However, the PSA test is not diagnostic. A raised level of PSA can be caused by other non-related factors such as benign prostate hyperplasia (BPH) and prostatitis. It is not specific to the disease state and is unable to indicate risk of death (Frankel et al., 2003). Clinical decisions cannot be informed by the PSA screen alone. The PSA test is unable to distinguish between malignant and nonmalignant forms or predict how a lesion may progress. Furthermore, not all prostate cancers give rise to an increase in serum PSA concentrations. Indeed 85% of men with raised PSA levels who undergo radical treatment (prostatectomy) do so without prospective benefit (Frankel et al., 2003).

Accordingly, there is a need for more reliable methods of diagnosing prostate cancer or its precursor, and for monitoring the disease over time (active monitoring), particularly at an early stage so that treatment options remain open. There is also a need for markers useful in determining patient status.

It is therefore an object of the invention to provide a marker useful in determining the prostate cancer status of a patient, or which at least provides the public with a useful choice.

It is a further object of the present invention to provide methods for diagnosing and/or prognosing prostate cancer or prostatic intraepithelial neoplasia or a predisposition thereto.

### SUMMARY OF THE INVENTION

In a first aspect, the invention provides an isolated nucleic acid molecule comprising the sequence of SEQ ID NO:3 or a functionally equivalent variant or fragment thereof, or a sequence which hybridises under stringent conditions to SEQ ID NO:3, or the variant or fragment thereof.

Preferably hybridisation is under stringent conditions.

In a further aspect, the invention provides an isolated nucleic acid molecule comprising an at least 10 nucleotide fragment of the nucleic acid sequence above, preferably SEQ ID NO:3, or a complement thereof, that hybridizes under stringent conditions to:
(a) a nucleic acid sequence above, preferably SEQ ID NO:3 or a complement thereof;
(b) the full-length coding sequence of the cDNA corresponding to a nucleic acid sequence above or a complement thereof;
(c) a reverse complement of (a) or (b).

The nucleic acid molecule may be at least 20, at least 30, at least 40, at least 50 nucleotides, at least 60 nucleotides, at least 70 nucleotides, at least 80 nucleotides, at least 90 nucleotides, or preferably is at least 100 nucleotides.

In a further aspect, the invention provides a genetic construct which comprises a nucleic acid molecule of the invention.

Preferably, the constructs are expression constructs as defined herein.

The invention further provides a vector which comprises a genetic construct of the invention.

The invention also provides a host cell which comprises a genetic construct or vector of the invention.

Also provided by the invention is an isolated polypeptide encoded by a nucleic acid molecule of the invention. Preferably, the polypeptide is at least 5 amino acids in length.

The invention also provides an isolated polypeptide comprising a sequence of (a) SEQ ID NO:9, (b) SEQ ID:10, (c) SEQ ID NO:11, (d) SEQ ID NO:12, (e) SEQ ID NO:13, or (f) SEQ ID NO;14; or a functionally equivalent variant or fragment of (a), (b), (c), (d), (e) or (f), or a sequence which hybridises under stringent conditions to any of (a), (b), (c), (d), (e) or (f).

In a further aspect, the invention provides a method for the recombinant production of a polypeptide of the invention, the method comprising the steps of:
(a) culturing a host cell comprising a genetic construct of the invention, such as an expression construct defined herein, capable of expressing a polypeptide of the invention;
(b) selecting cells expressing the polypeptide of the invention;
(c) separating the expressed polypeptide from the cells; and optionally
(d) purifying the expressed polypeptide.

As a pre-step the method may comprise transfecting the host cells with the construct.

The invention also provides an antibody which specifically binds to a polypeptide encoded by a nucleic acid molecule of the invention, or a functionally equivalent variant or fragment of the polypeptide.

Preferably, the antibody is a polyclonal, monoclonal, single chain or humanized antibody, or immunologically active fragment thereof.

In a further aspect, the invention provides an array comprising one or more nucleic acid sequences which bind PSPU 43 (SEQ ID NO:3).

The invention also provides an array comprising one or more nucleic acid sequences of the invention.

Preferably, the array further comprises one or more nucleic acid sequences which bind one or more of transgelin 1 (SEQ ID NO:7), transgelin 2 (SEQ ID NO:8), PCA3 (SEQ ID NO:6), and PSA (SEQ ID NO:5).

The invention also provides a method of screening for a compound that alters the expression of a nucleic acid molecule of the invention, preferably PSPU 43 (SEQ ID NO:3), the method comprising the steps of:
(a) contacting a test cell that expresses the nucleic acid with a test compound;
(b) determining the expression level of the nucleic acid; and
(c) selecting the compound that alters the expression level compared to that in the absence of the test compound.

Further provided is a method of screening for a compound that alters the activity of a nucleic acid of any one of the invention, preferably PSPU 43 (SEQ ID NO:3) marker, the method comprising:
(a) contacting a test compound with a peptide encoded by the nucleic acid molecule;
(b) detecting the biological activity of the peptide; and either:
(c) selecting the compound that alters the biological activity of the peptide in comparison with the biological activity detected in the absence of the compound; or
(d) selecting the compound that binds to the peptide.

The invention also provides a compound that alters expression or activity of a nucleic acid molecule of the invention, preferably PSPU 43 (SEQ ID NO:3) when selected by the screening methods of the invention.

In yet a further aspect, the invention provides a composition comprising a pharmaceutically effective amount of a compound selected by a screening method of the invention.

The invention also relates to a use of a compound of the invention in the preparation of a medicament for the treatment of PIN or PRC.

A PIN or PRC expression profile, comprising a pattern of marker expression including a nucleic acid molecule of the invention, preferably PSPU 43 (SEQ ID NO:3), is also provided by the present invention. Preferably, the profile further comprises one or more markers selected from transgelin 1 (SEQ ID NO:7), transgelin 2 (SEQ ID NO:8), PCA 3 (SEQ ID NO:6), and PSA (SEQ ID NO:5).

The invention in a further aspect, provides a method of treating or preventing PIN or PRC in a patient, the method comprising altering the expression level of a nucleic acid molecule of the invention, preferably PSPU 43 (SEQ ID NO:3) in the patient, or the activity of a peptide encoded by the marker. This may be by promoting expression, or administration of a composition comprising a polypeptide encoded by the nucleic acid molecule such as PSPU 43. Alternatively, this may be by inhibiting expression. Whether promotion or inhibition of expression levels is appropriate will depend on whether polypeptides encoded by the nucleic acid molecules and PSPU 43 are being over- or under-expressed. Without wishing to be bound by theory, both over- and under-expression are believed to be possible at this time.

Preferably, the polypeptides encoded by the nucleic acid molecules of the invention are overexpressed, and expression is inhibited by administering an antisense composition to the patient, the composition comprising one or more nucleotide sequences antisense to a nucleic acid molecule of the invention, preferably antisense to PSPU 43 (SEQ ID NO:3).

In another embodiment expression is inhibited by administering a siRNA composition to the patient. The composition reduces the expression of a nucleic acid molecule of the invention, preferably PSPU 43 (SEQ ID NO:3).

In another embodiment expression is inhibited by administering a ribozyme composition to the patient.

Expression may also be inhibited by administering an antibody or active antibody fragment which specifically binds to a nucleic acid molecule of the invention, preferably to PSPU 43 (SEQ ID NO:3). The active fragment is preferably an immunologically active fragment.

In one embodiment the composition administered is a vaccine.

The invention also relates to an antisense-oligonucleotide, ribozyme or siRNA against a nucleic acid molecule of the invention, preferably PSPU 43 (SEQ ID NO:3). The sequences are useful in the above method.

The invention also provides a method of treating or preventing PIN or PRC in a patient wherein a polypeptide of the invention, for example a PSPU 43 encoded polypeptide, is under-expressed, the method comprising administering to said patient a composition comprising the under-expressed polypeptide encoded by a nucleic acid molecule of the invention, such as PSPU 43 (SEQ ID NO:3), or an active variant or fragment of the polypeptide.

In a still further aspect, the invention provides a method of treating or preventing PIN or PRC in a patient, the method comprising administering to said patient a compound that alters the expression or activity of a polypeptide of the invention, preferably a polypeptide encoded by PSPU 43 (SEQ ID NO:3).

In a still further aspect, the invention provides a method of treating or preventing PIN or PRC in a patient wherein a nucleic acid molecule of the invention, preferably PSPU 43 (SEQ ID NO:3) is over-expressed, the method comprising administering to said patient a compound that decreases the expression or activity of a polypeptide of the invention, preferably a polypeptide encoded by PSPU 43.

The invention also provides, a composition comprising a pharmaceutically effective amount of nucleic acid molecules of the invention, preferably PSPU 43 (SEQ ID NO:3) or a polypeptide encoded by same.

Further provided by the invention is a composition comprising a pharmaceutically effective amount of an antisense-oligonucleotide, ribozyme or small interfering RNA against a nucleic acid molecule of the invention, preferably PSPU 43 (SEQ ID NO:3).

The composition may comprise two or more antisense-oligonucleotides, ribozymes or siRNAs against the nucleic acid molecule.

In another aspect, the invention provides a composition comprising a pharmaceutically effective amount of an antibody or fragment thereof that specifically binds to a polypeptide of the invention, preferably a polypeptide encoded by PSPU 43 (SEQ ID NO:3) marker.

The invention also provides a method of treating PIN or PRC in a patient, the method comprising administering an effective amotmt of a compound of the invention or a composition of the invention to a patient in need thereof.

Also provided by the invention is use of a nucleic acid molecule of the invention, preferably PSPU 43 (SEQ ID NO:3), or polypeptide encoded by same in the preparation of a medicament for treating or preventing PIN or PRC in a patient.

The invention also provides an assay for detecting the presence of a nucleic acid molecule of the invention, preferably PSPU43 (SEQ ID NO:3) in a sample, the method comprising:
(a) contacting the sample with a nucleotide probe which hydridises to the nucleic acid sequence of the invention, preferably PSPU43 (SEQ ID NO:3) under stringent hybridisation conditions; and
(b) detecting the presence of a hybridisation complex in the sample.

Preferably, the probe is a labelled probe, commonly a fluorescently labelled probe. In one embodiment the probe is a complement of SEQ ID NO:3.

The invention also provides a method of determining the level of expression of a nucleic acid molecule of the invention, preferably PSPU 43 (SEQ ID NO:3), in a patient sample, the method comprising direct or indirect measurement of the nucleic acid molecule.

Conveniently, the nucleic acid molecule is measured by employing same in an in situ hybridisation or RT-PCR analysis.

The invention also relates to a method of determining the level of expression of a nucleic acid molecule of the invention, preferably PSPU 43 (SEQ ID NO:3) in a sample, the method comprising:
(a) amplifying the DNA sequence of the nucleic acid molecule or a complement thereof; or
(b) amplifying the cDNA sequence of the nucleic acid molecule or a complement thereof; and
(c) measuring the level of one or more of DNA, cDNA or RNA in said sample.

The invention also provides an assay for detecting the presence in a patient sample of a polypeptide of the invention the method comprising:

(a) contacting the sample with an antibody of the invention; and
(b) detecting the presence of bound polypeptide in the sample.

Preferably, the antibody is detectably labelled.

In a further aspect, the invention relates to a method of diagnosing prostatic intraepithelial neoplasia (PIN), prostate cancer (PRC) status or a predisposition to developing PIN or PRC in a patient, the method comprising determining the expression level of a nucleic acid molecule of the invention, preferably PSPU 43 (SEQ ID NO:3) in a patient sample, wherein an alteration in expression level compared to a control level of said nucleic acid molecule indicates that the patient has PIN, PRC, or is at risk of developing PIN or PRC.

Most usually, the alteration in expression level is at least 10% above the normal control level. The control level is conveniently the expression level of PSPU 43 measured in normal prostate.

The sample may comprise normal prostate cells, or PIN or PRC cells, and preferably epithelial cells from normal prostate, PIN or prostate cancer tumour.

The invention also provides a method of testing for prostatic intraepithelial neoplasia (PIN) and prostate cancer (PRC) status in a patient, the method comprising determining the expression level of a nucleic acid molecule of the invention, preferably PSPU 43 (SEQ ID NO:3) in a patient sample, wherein an increase in expression level compared to a control level of said molecule indicates that the patient has PIN, PRC status or is at risk of developing PIN or PRC.

In another aspect, the invention relates to a method of monitoring response to treatment of PIN or PRC in a subject, the method comprising determining the expression level of a nucleic acid molecule of the invention, preferably PSPU 43 (SEQ ID NO:3) in a patient sample, and comparing the level of said PSPU 43 (SEQ ID NO:3) to a control level, wherein a statistically significant change in the determined level from the control level is indicative of a response to the treatment.

Preferably, these determining, testing and monitoring methods further comprise determining the level of one or more additional markers of PIN or PRC and comparing the levels to marker levels from a control, wherein a significant deviation in the levels from a control level, together with a statistically significant increase in the level of a nucleic acid molecule of the invention, preferably PSPU 43 (SEQ ID NO:3) is indicative of PRC or PIN, or can be used to monitor PIN or PRC.

The additional markers may be one or more markers selected from transgelin 1 (SEQ ID NO:7), transgelin 2 (SEQ ID NO:8), PCA 3 (SEQ ID NO:6) and PSA (SEQ ID NO:5).

The invention also provides a kit for detecting the presence of a nucleic acid molecule of the invention, preferably PSPU 43 (SEQ ID NO:3), in a sample, the kit comprising at least one container with the nucleic acid of the invention contained therein.

The invention also provides a kit comprising one or more detection reagents which bind a nucleic acid molecule of the invention, or a polypeptide of the invention.

In one embodiment the kit further comprises one or more of:

(a) nucleic acid encoding transgelin 1 (SEQ ID NO:7) or a complement thereof;
(b) nucleic acid encoding transgelin 2 (SEQ ID NO:8) or a complement thereof;
(c) nucleic acid encoding PCA3 (SEQ ID NO:6) or a complement thereof; and
(d) nucleic acid encoding PSA (SEQ ID NO:5) or a complement thereof.

In another aspect, the invention relates to a diagnostic, testing or monitoring method of the invention in which transgelin 2 (SEQ ID NO:8) is used as a reference marker.

The invention also relates to the use of transgelin 2 (SEQ ID NO:8) as a reference marker in the diagnostic, testing and monitoring methods of the invention.

Also provided is a non-human animal having a genome wherein the nucleic acid sequence PSPU 43 (SEQ ID NO:3) is altered, disrupted, eliminated or added.

Preferably the animal is a mouse.

### DESCRIPTION OF THE DRAWINGS

The invention will now be described with reference to the figures in accompanying drawings in which:

FIG. 1: Shows the consensus sequence for Pspu43. The consensus sequence is generated from contigs of EST's comprising UniGene cluster Hs.161160;

FIG. 2: Shows the dissociation curves for SYBR Green qPCR assays using primer sets for Pspu1, Pspu2, Pspu8, Pspu43, T1 and T2. The cDNA template used was generated from the PC3 cell line;

FIG. 3: Shows the qPCR efficiency of each primer and probe/primer combination used to test for marker expression in prostate tissue. Standard curves used a universal reference cDNA as template;

FIG. 4: Shows the average raw CT values from cDNA templates generated from matched tissue pairs. Each error bar indicates +/−1 standard deviation calculated from duplicate qPCR reactions;

FIG. 5: Shows the relative amount of cDNA per sample for each marker corrected for genomic DNA contamination. Relative quantity was determined from the average CT and the line of best fit calculated for the standard curve run for each marker. A similar calculation was performed for qPCR on RNA only templates (no RT reaction) and the relative quantity values for this reaction subtrated from the values determined for cDNA templates; and

FIG. 6: Shows the translation in six reading frames of nucleotide sequence Pspu43. The single letter amino acid code has been used. —represents a stop codon.

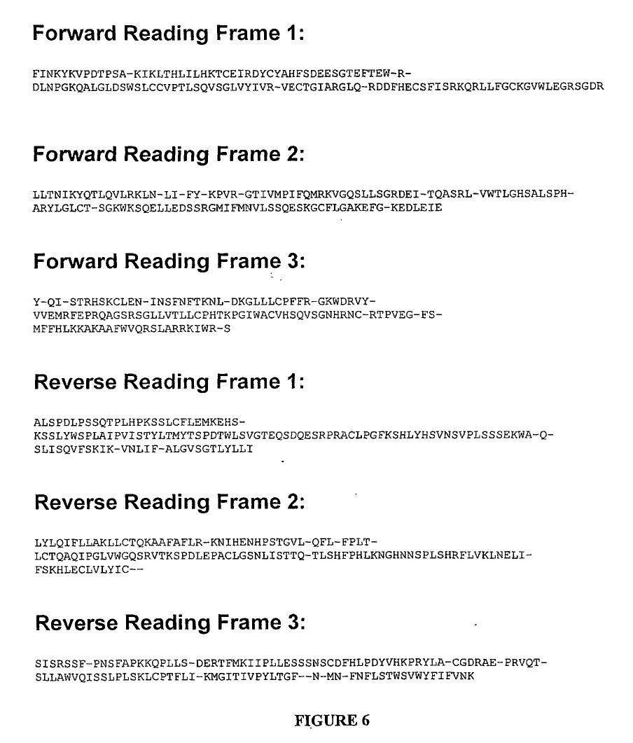

### DEFINITIONS

The term “comprising” as used in this specification and claims means “consisting at least in part of”, that is to say when interpreting statements in this specification and claims which include the term, the features, prefaced by that term in each statement, all need to be present but other features can also be present. Related terms such as “comprise” and “comprised” are to be interpreted in a similar manner.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

The term “marker” as used herein refers to a segment of DNA with an identifiable physical location on a chromosome. A marker may be a gene or other identifiable nucleic acid sequence, such as an open reading frame, a portion of an intron or an intergenic genomic DNA Segment

A “control level” of a marker as used herein refers to a level of expression detected in a sample from a normal healthy individual, or a level determined based on a population of individuals not known to be suffering from PRC or PIN. The control level may be a single expression pattern derived from a single reference population or may be a plurality of expression patterns. For example, the control level can be a database of expression patterns from previously tested cells. Another example may be a ratiometric measure between a reference marker (e.g. transgelin 2) and a marker of the invention. Alternatively, the control level may be one or more readings or the mean of such readings taken from the same patient at an earlier time.

The term “polynucleotide(s),” as used herein, means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments Reference to a nucleic acid molecule is to be similarly understood.

“Antisense” as used herein generally means DNA or RNA or a combination of same that is complementary to at least a portion of an mRNA molecule encoding a polypeptide of the invention, and capable of interfering with a post-transcriptional event such as mRNA translation.

A “fragment” of a polynucleotide sequence provided herein is a subsequence of contiguous nucleotides that is capable of specific hybridization to a target of interest, e.g., a sequence that is at least 10 nucleotides in length. The fragments of the invention comprise 10, preferably 15 nucleotides, preferably at least 20 nucleotides, more preferably at least 30 nucleotides, more preferably at least 40 nucleotides, more preferably at least 50 nucleotides and most preferably at least 60 nucleotides of contiguous nucleotides of a polynucleotide of the invention. A fragment of a polynucleotide sequence can be used in antisense, gene silencing, triple helix or ribozyme technology, or as a primer, a probe, included in a microarray, or used in polynucleotide-based selection methods of the invention.

The term “patient” as used herein is preferably a mammalian patient and includes humans, and non-human mammals such as cats, dogs, horses, cows, sheep, deer, mice, possum and primates (including gorillas, rhesus monkeys and chimpanzees) and other domestic farm or zoo animals. Preferably, the mammal is human.

The term “treat”, “treating” or “treatment” and “preventing” refer to therapeutic and phrophylactic measures which stop, reverse or lessen prostate cancer or PIN. The patient shows observable or measurable (statistically significant) reduction in one or more of: number of cancer or PIN cells; tumour size; symptoms associated with the cancer or PIN; inhibition of: tumour size; tumour growth; metastasis; improvement in quality of life.

A “patient sample” as used herein means a biological sample derived from a patient to be screened. The biological sample may be any suitable sample known in the art in which the expression of the selected markers can be detected. Included are individual cells and cell populations obtained from bodily tissues or fluids. Examples of suitable body fluids to be tested are plasma, prostate massage fluid, blood, semen, lymph and urine.

Preferably, the sample to be tested comprises epithelial cells derived from tissue that is known or suspected to exhibit PIN or PRC, most usually prostate tissue. Samples from healthy individuals may also be tested. A normal healthy individual is one with no clinical symptoms of PIN or PRC or benign prostate hypoplasia (BPH) and preferably under 30 years of age. Alternately, normal healthy cells from normal regions of a prostate biopsy may be used as controls in the methods.

The term “primer” refers to a short polynucleotide, usually having a free 3′OH group, that is hybridized to a template and used for priming polymerization of a polynucleotide complementary to the target.

The term “probe” refers to a short polynucleotide that is used to detect a polynucleotide sequence, that is complementary to the probe, in a hybridization-based assay. The probe may consist of a “fragment” of a polynucleotide as defined herein.

The term “polypeptide”, as used herein, encompasses amino acid chains of any length, but preferably at least 5 amino acids, preferably at least 10, preferably at least 15, preferably at least 20, preferably at least 25, preferably at least 30, preferably at least 40, preferably at least 50, preferably at least 60, preferably at least 70, preferably at least 80, preferably at least 90, preferably at least 100, preferably at least 110, preferably at least 120, preferably at least 125 amino acids, and including full-length proteins, in which amino acid residues are linked by covalent peptide bonds. Polypeptides of the present invention may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

A “fragment” of a polypeptide is a subsequence of the polypeptide that performs a function that is required for the biological activity and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques. The polynucleotide or polypeptide sequences may be prepared by at least one purification step.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polypeptides and polynucleotides possess biological activities that are the same or similar to those of the inventive polypeptides or polynucleotides. The term “variant” with reference to polynucleotides and polypeptides encompasses-all forms of polynucleotides and polypeptides as defined herein.

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence of the present invention. Identity is found over a comparison window of at least 20 nucleotide positions, preferably at least 50 nucleotide positions, more preferably at least 100 nucleotide positions, and most preferably over the entire length of a polynucleotide of the invention.

Polynucleotide sequence identity may be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants also encompass those which exhibit a similarity to one or more of the specifically identified sequences that is likely to preserve the functional equivalence of those sequences and which could not reasonably be expected to have occurred by random chance. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻⁵ more preferably less than 1×10⁻⁶, more preferably less than 1×10⁻⁹, more preferably less than 1×10⁻¹², more preferably less than 1×10⁻¹⁵, more preferably less than 1×10⁻¹⁸ and most preferably less than 1×10⁻²¹ when compared with any one of the specifically identified sequences.

Use of BLASTN is preferred for use in the determination of sequence identity for polynucleotide variants according to the present invention.

The identity of polynucleotide sequences may be examined using the following UNIX command line parameters:

bl2seq-i nucleotideseq1-j nucleotideseq2-F F-p blastn The parameter-F F turns off filtering of low complexity sections. The parameter-p selects the appropriate algorithm for the pair of sequences. The bl2seq program reports sequence identity as both the number and percentage of identical nucleotides in a line “Identities=”.

Polynucleotide sequence identity and similarity can also be determined in the following manner. The subject polynucleotide sequence is compared to a candidate polynucleotide sequence using sequence alignment algorithms and sequence similarity search tools such as in Genbank, EMBL, Swiss-PROT and other databases. Nucleic Acids Res 29:1-10 and 11-16, 2001 provides examples of online resources. BLASTN (from the BLAST suite of programs, version 2.2.13 Mar. 2007 in bl2seq (Tatiana A. et al, FEMS Microbiol Lett. 174:247-250 (1999), Altschul et al., Nuc. Acis Res 25:3389-3402, (1997)), which is publicly available from NCBI (ftp://ftp.ncbi.nih.gov/blast/) or from NCB1 at Bethesda, Md., USA. The default parameters of bl2seq are utilized except that filtering of low complexity parts should be turned off.

Alternatively, variant polynucleotides hybridize to the specified polynucleotide sequence, or a complement thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing, incorporated herein by reference). Tm for polynucleotide molecules greater than about 100 bases can be calculated by the formula Tm=81.5+0.41% (G+C-log(Na+) (Sambrook et. al., Eds, 1987, Molecular Cloning, A Laboratory Manual, 2nd Ed. Cold Spring Harbor Press; Bolton and McCarthy, 1962, PNAS 84:1390). Typical stringent conditions for a polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

In one embodiment stringent conditions use 50% formamide, 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulphate at 42° C., with washes at 42° C. in 0.2×SSC and 50% formamide at 55° C., followed by a wash comprising of 0.1×SSC containing EDTA at 55° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the bl2seq program via the tblastx algorithm as described above.

The term “antisense-oligonucleotides” as used herein encompasses both nucleotides that are entirely complementary to the target sequence and those having a mismatch of one or more nucleotides, so long as the antisense-oligonucleotides can specifically hybridize to the target sequence. For example, the antisense-oligonucleotides of the present invention include polynucleotides that have an identity of at least 70% or higher, preferably at least 75% or higher, at least 76%, at least 77%, at least 78%, at least 79%, at least 80% or higher, more preferably at least 81%, at least 82%, at least 83%, at least 84%, at least 85%, at least 86%, at least 87%, at least 88%, at least 89%, at least 90% or higher, even more preferably 91%, 92%, 93%, 94%, 95%, 96%, 97%, or 99% over a span of at least 15, at least 20, at least 30, at least 40, at least 50, preferably 75, preferably 100, more preferably 200 contiguous nucleotides, or the full length of a nucleic acid sequence of the invention, preferably of PSPU 43 (SEQ ID NO:3). Algorithms known in the art as discussed above can be used to determine the identity. Furthermore, derivatives or modified products of the antisense-oligonucleotides can also be used as antisense-oligonucleotides in the present invention.

The term “variant” with reference to polypeptides encompasses naturally occurring, recombinantly and synthetically produced polypeptides. Variant polypeptide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence of the present invention. Identity is found over a comparison window of at least 20 amino acid positions, preferably at least 50 amino acid positions, more preferably at least 100 amino acid positions, and most preferably over the entire length of a polypeptide of the invention.

Polypeptide variants also encompass those which exhibit a similarity to one or more of the specifically identified sequences that is likely to preserve the functional equivalence of those sequences and which could not reasonably be expected to have occurred by random chance.

Polypeptide sequence identity and similarity can be determined in the following manner. The subject polypeptide sequence is compared to a candidate polypeptide sequence using BLASTP (from the BLAST suite of programs, version 2.2.13 May 2007) in bl2seq, which is publicly available from NCBI (ftp://ftp.ncbi.nih.gov/blast/). The default parameters of bl2seq are utilized except that filtering of low complexity regions should be turned off.

The similarity of polypeptide sequences may be examined using the following UNIX command line parameters:
bl2seq-i peptideseq1-j peptideseq2-F F-p blastp

Variant polypeptide sequences preferably exhibit an E value of less than 1×10⁻⁵, more preferably less than 1×10⁻⁶, more preferably less than 1×10⁻⁹, more preferably less than 1×10⁻¹², more preferably less than 1×10⁻¹⁵, more preferably less than 1×10⁻¹⁸ and most preferably less than 1×10⁻²¹ when compared with any one of the specifically identified sequences.

The parameter-F F turns off filtering of low complexity sections. The parameter-p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Use of BLASTP as described above is preferred for use in the determination of polypeptide variants according to the present invention.

Conservative substitutions of one or several amino acids of a described polypeptide sequence without significantly altering its biological activity are also included in the invention. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative substitutions can be taken from Table 1 below.

TABLE 1

| Original | Exemplary |
|---|---|
| Residue | Substitutions |
| Ala (A) | val; leu; ile; gly |
| Arg (R) | lys; gln; asn |
| Asn (N) | gln; his; lys; arg |
| Asp (D) | glu |
| Cys (C) | ser |
| Gln (Q) | asn; his |
| Glu (E) | asp |
| Gly (G) | pro; ala |
| His (H) | asn; gln; lys; arg |
| Ile (I) | leu; val; met; ala; |
| | phe; norleucine |
| Leu (L) | norleucine; ile; |
| | val; met; ala; phe |
| Lys (K) | arg; gln; asn |
| Met (M) | leu; phe; ile |
| Phe (F) | leu; val; ile; ala; |
| | tyr |
| Pro (P) | ala; gly |
| Ser (S) | thr |
| Thr (T) | ser |
| Trp (W) | tyr; phe |
| Tyr (Y) | trp; phe; thr; ser |
| Val (V) | ile; leu; met; phe; |
| | ala; norleucine |

Naturally occurring residues are divided into groups based on common side-chain properties:

(1) hydrophobic: norleucine, met, ala, val, leu, ile;
(2) neutral hydrophilic: cys, ser, thr;
(3) acidic: asp, glu;
(4) basic: asn, gln, his, lys, arg:
(5) residues that influence chain orientation: gly, pro; and
(6) aromatic: tip, tyr, phe.

Non-conservative substitutions will entail exchanging a member of one of these classes for a member of another class.

Other variants include peptides with modifications which influence peptide stability. Such analogs may contain, for example, one or more non-peptide bonds (which replace the peptide bonds) in the peptide sequence. Also included are analogs that include residues other than naturally occurring L-amino acids, e.g. D-amino acids or non-naturally occurring synthetic amino acids, e.g. beta or gamma amino acids and cyclic analogs.

Substitutions, deletions, additions or insertions may be made by mutagenesis methods known in the art. A skilled artisan will be aware of methods for making phenotypically silent amino acid substitutions (see, e.g., Bowie et al., 1990, Science 247, 1306).

Also included within the polypeptides of the invention are those which have been modified during or after synthesis for example by biotinylation, benzylation, glycosylation, phosphorylation, amidation, by derivatization using blocking/protecting groups and the like. Such modifications may increase stability or activity of the polypeptide.

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences and/or other regulatory elements.

“Operably-linked” means that the sequence to be expressed is placed under the control of regulatory elements that include promoters, transcription control sequences, translation control sequences, origins of replication, tissue-specific regulatory elements, temporal regulatory elements, enhancers, polyadenylation signals, repressors and terminators.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors.

The terms “to alter expression of” and “altered expression” of a polynucleotide or polypeptide of the invention, are intended to encompass the situation where genomic DNA corresponding to a polynucleotide of the invention is modified thus leading to altered expression of a polynucleotide or polypeptide of the invention. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

### DETAILED DESCRIPTION OF THE INVENTION

The applicants have identified a novel marker for prostate cancer or PIN using a new bioinformatics approach to mine sequenced prostate cDNA libraries. Data-panning is a technique which determines the degree of specificity transcripts have to a given tissue. This method utilises the UniGene database (www.ncbi.nlm.nih.gov/UniGene). ESTs within a UniGene cluster are assessed for library of origin and a tally of those from the specified tissue is kept. This tally is expressed as a percentage of the total number of EST's in the UniGene cluster. The higher the percentage the fewer instances of that transcript being detected in tissues other than those specified. This approach has advantages over other technologies such as cDNA microarrays (Carlisle et al., 2000), due to unbiased gene selection, and greater discriminatory power in identifying differences between disease states. Previous attempts to profile gene expression in prostate cancer have employed methods that are limited in the number of expressed sequence tags analysed (Huang et al., 1999) or biased in gene selection (Carlisle et al., 2000).

From this analysis the applicants have identified a new marker whose expression is believed to alter with the progression of prostate cancer or PIN. This marker may also be a promising new target for the development of drugs to treat prostate cancer or PIN.

The marker is listed in Table 1 below, and the full sequence given in the sequence listing. The expression level of the markers is altered in prostate cancer patients. For convenience the marker is referred to herein as prostate secific unigene (PSPU) marker. The marker may be a DNA or RNA sequence, gene or chromosomal fragment. Any corresponding polypeptides encoded by genes are referred to as PSPU polypeptides or PSPU proteins.

Marker Implicated in Prostate Cancer or Pin

| Name | Unigene # | SEQ ID NO: | % Enrichment | Tissues |
|---|---|---|---|---|
| PSPU 43 | 161160 | 3 | 83 | Prostate, Other |

The nucleic acid molecules of the invention or otherwise described here can be isolated from tissue using a variety of techniques known to those of ordinary skill in the art. By way of example, such polynucleotides can be isolated through use of the polymerase chain reaction (PCR) described in Mullis et al., Eds. 1994 The Polymerase Chain Reaction, Birkhauser. The nucleic acid molecules of the invention can be amplified using primers, as defined herein, derived from the polynucleotide sequences of the invention.

Further methods for isolating polynucleotides include use of all, or portions of, the polynucleotide of the invention, particularly a polynucleotide having the sequence set forth in SEQ ID NO:3 as hybridization probes. The technique of hybridizing labeled polynucleotide probes to polynucleotides immobilized on solid supports such as nitrocellulose filters or nylon membranes, can be used to screen genomic or cDNA libraries. Similarly, probes may be coupled to beads and hybridized to the target sequence. Isolation can be effected using known art protocols such as magnetic separation. Exemplary stringent hybridization and wash conditions are as given above.

Polynucleotide fragments may be produced by techniques well-known in the art such as restriction endonuclease digestion and oligonucleotide synthesis.

Accordingly, in a first aspect the invention provides an isolated nucleic acid comprising SEQ ID NO:3, a functionally equivalent variant or fragment of same, or a sequence which hybridizes to any of these under stringent conditions.

In a further aspect, the invention provides an isolated nucleic acid molecule consisting of an at least 10 nucleotide fragment of the nucleic acid sequence of the invention, preferably of SEQ ID NO:3 or a complement thereof, that hybridizes under stringent conditions to:
(a) the nucleic acid sequence of the invention, preferably SEQ ID NO:3 or a complement thereof;
(b) the full-length coding sequence of the cDNA corresponding to a nucleic acid sequence of the invention or a complement thereof;
(c) a reverse complement of (a) or (b).

Stringent conditions are as discussed above.

The nucleic acid molecule may be at least 20 nucleotides, at least 30 nucleotides, at least 40 nucleotides, at least 50 nucleotides, at least 60 nucleotides, at least 70 nucleotides, at least 80 nucleotides, at least 90 nucleotides, or preferably is at least 100 nucleotides.

A partial polynucleotide sequence may be used as a probe, in methods well-known in the art to identify the corresponding full length polynucleotide sequence in a sample. Such methods include PCR-based methods, 5′RACE (Methods Enzymol. 218: 340-56 (1993); Sambrook et al., Supra) and hybridization-based method, computer/database-based methods. Detectable labels such as radioisotopes, fluorescent, chemiluminescent and bioluminescent labels may be used to facilitate detection. Inverse PCR also permits acquisition of unknown sequences, flanking the polynucleotide sequences disclosed herein, starting with primers based on a known region (Triglia et al., Nucleic Acids Res 16, 8186, (1998)) The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Supra). Primers and primer pairs which allow amplification of polynucleotides of the invention, also form a further aspect of this invention.

Variants (including orthologues) may be identified by the methods described. Variant polynucleotides may be identified using PCR-based methods (Mullis et al., Eds. 1994 The Polymerase Chain Reaction, Birkhauser). Typically, the polynucleotide sequence of a primer, useful to amplify variants of polynucleotide molecules by PCR, may be based on a sequence encoding a conserved region of the corresponding amino acid sequence.

Further methods for identifying variant polynucleotides include use of all, or portions of, the specified polynucleotides as hybridization probes to screen genomic or cDNA libraries as described above. Typically probes based on a sequence encoding a conserved region of the corresponding amino acid sequence may be used. Hybridisation conditions may also be less stringent than those used when screening for sequences identical to the probe.

The variant sequences, including both polynucleotide and polypeptide variants, may also be identified by the computer-based methods discussed above.

Multiple sequence alignments of a group of related sequences can be carried out with CLUSTALW (Thompson, et al., Nucleic Acids Research, 22:4673-4680 (1994), http://www-igbmc.u-strasbg.fr/BioInfo/ClustalW/Top.html) or T-COFFEE (Cedric Notredame et al., J. Mol. Biol. 302: 205-217 (2000))) or PILEUP, which uses progressive, pairwise alignments. (Feng et al., J. Mol. Evol. 25, 351 (1987)).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch et al., Nucleic Acids Res. 22, 3583 (1994); Hofmann et al., Nucleic Acids Res. 27, 215 (1999)) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., Nucleic Acids Res. 30, 235 (2002)). Prosearch is a tool that can search SWISS-PROT and EMBL databases with a given sequence pattern or signature.

Proteins can be classified according to their sequence relatedness to other proteins in the same genome (paralogues) or a different genome (orthologues). Orthologous genes are genes that evolved by speciation from a common ancestral gene and normally retain the same function as they evolve. Paralogous genes are genes that are duplicated within a genome and genes may acquire new specificities or modified functions which may be related to the original one. Phylogenetic analysis methods are reviewed in Tatusov et al., Science 278, 631-637, 1997).

In addition to the computer/database methods described above, polypeptide variants may be identified by physical methods, for example by screening expression libraries using antibodies raised against polypeptides of the invention (Sambrook et al., Molecular Cloning: A Laboratory Manual, 2nd Ed. Cold Spring Harbor Press, 1987) by recombinant DNA techniques also described by Sambrook et al. or by identifying polypeptides from natural sources with the aid of such antibodies.

Polypeptides, including variant polypeptides, may be prepared using peptide synthesis methods well known in the art such as direct peptide synthesis using solid phase techniques (e.g. Merrifield, 1963, in J. Am Chem. Soc. 85, 2149; Stewart et al., 1969, in Solid-Phase Peptide Synthesis, WH Freeman Co, San Francisco Calif.; Matteucci et al. J. Am. Chem. Soc. 103:3185-3191, 1981) or automated synthesis, for example using a Synthesiser from Applied Biosystems (California, USA). Mutated forms of the polypeptides may also be produced using synthetic methods such as site-specific mutagenesis of the DNA encoding the amino acid sequence as described by Adelmen et al; DNA 2, 183 (1983).

The polypeptides and variant polypeptides may also be isolated or purified from natural sources using a variety of techniques that are well known in the art (e.g. Deutscher, 1990, Ed, Methods in Enzymology, Vol. 182, Guide to Protein Purification). Technologies include HPLC, ion-exchange chromatography, and immunochromatography but are not limited thereto.

Alternatively the polypeptides and variant polypeptides may be expressed recombinantly in suitable host cells and separated from the cells as discussed below. The polypeptides and variants have utility in generating antibodies, and generating ligands amongst other uses.

Accordingly, the invention also provides isolated polypeptides encoded by a nucleic acid molecule of the invention.

Specific polypeptides of the invention include polypeptides having the amino acid sequences of SEQ ID NO:9, SEQ ID NO:10, SEQ ID NO:11, SEQ ID NO:12, SEQ ID NO:13 and SEQ ID NO:14 all as set forth in the accompanying sequence listing. Also contemplated are functional equivalent variants and fragments of these polypeptides as defined herein and sequences, which hybridise to those sequences, under stringent conditions.

The genetic constructs described herein may comprise one or more of the disclosed polynucleotide sequences and/or polynucleotides encoding the disclosed polypeptides, of the invention and may be useful for transforming, for example, bacterial, fungal, insect, mammalian or plant organisms. The genetic constructs of the invention are intended to include expression constructs as herein defined. Included are vectors (such as pBR322, pUC18, pU19, Mp18, Mp19, ColE1, PCR1 and pKRC), phages (such as lambda gt10), and M13 plasmids (such as pBR322, pACYC184, pT127, RP4, p1J101, SV40 and BPV), cosmids, YACS, BACs shuttle vectors such as pSA3, PAT28 transposons (such as described in U.S. Pat. No. 5,792,294) and the like.

The constructs may conveniently include a selection gene or selectable marker. Typically an antibiotic resistance marker such as ampicillin, methotrexate, or tetracycline is used.

Promoters useful in the constructs include β. lactamase, alkaline phosphatase, tryptophan, and tac promoter systems which are all well known in the art. Yeast promoters include 3-phosphoglycerate kinase, enolase, hexokinase, pyruvate decarboxylase, glucokinase, and glyceraldehydrate-3-phosphanate dehydrogenase but are not limited thereto.

Enhancers may also be employed to act on the promoters to enhance transcription. Suitable enhancers for use herein include SV40 enhancer, cytomeglovirus early promoter enhancer, globin, albumin, insulin and the like.

Methods for producing and using genetic constructs and vectors are well known in the art and are described generally in Sambrook et al., (supra), and Ausubel et al., Current Protocols in Molecular Biology, Greene Publishing, 1987. Methods for transforming selected host cells with the vectors are also known, for example, the calcium chloride treatment described by Cohen, S N; PNAS 69, 2110, 1972.

Host cells comprising the genetic constructs and vectors described may be derived from prokaryotic or eukaryotic sources, for example yeast, bacteria, fungi, insect (eg baculovirus), animal, mammalian or plant organisms. Prokaryotes most commonly employed as host cells are strains of E. coli. Other prokaryotic hosts include Pseudomonas, Bacillus, Serratia, Klebsiella, Streptomyces, Listeria, Saccharomyces, Salmonella and Mycobacteria but are not limited thereto.

Eukaryotic cells for expression of recombinant protein include but are not limited to Vero cells, HeLa, CHO (Chinese Hamster ovary cells), 293, BHK cells, MDCK cells, and COS cells as well as prostate cancer cell lines such as PrEC, LNCaP, Du 145 and RWPE-2. The cells are available from ATCC, Virginia, USA.

Prokaryotic promoters compatible with expression of nucleic acid molecules of the invention include known art constitutive promoters (such as the int promoter of bacteriophage lamda and the bla promoter of the beta-lactamase gene sequence of pBR322) and regulatable promoters (such as lacZ, recA and gal). A ribosome binding site upstream of the PSPU 43 coding sequence is also required for expression.

Host cells comprising genetic constructs, such as expression constructs, are useful in methods for recombinant production of polypeptides. Such methods are well known in the art (see for example Sambrook et al. supra). The methods commonly involve the culture of host cells in an appropriate medium in conditions suitable for or conducive to, expression and selection of a polypeptide of the invention. Cells with a selectable marker may additionally be grown on medium appropriate for selection of host cells expressing a polypeptide of the invention. Transformed host cells expressing a polypeptide of the invention are selected and cultured under conditions suitable for expression of the polypeptide. The expressed recombinant polypeptide, may be separated and purified from the culture medium using methods well known in the art including ammonium sulfate precipitation, ion exchange chromatography, gel filtration, affinity chromatography, electrophoresis and the like (e.g. Deutscher, Ed, 1990, Methods in Enzymology, Vol 182, Guide to Protein Purification). Host cells may also be useful in methods for production of a product generated by an expressed polypeptide of the invention.

The invention also provides animal models. Host cells or animals that are predisposed to prostate cancer are useful for testing compounds which may be used to treat prostate cancer, or to identify compounds that may be implicated in causing the cancer. Animal models are particularly useful for testing purposes. Non-human patients as defined herein may be suitable animals to use. Preferably the animal is a rodent or rabbit. Rats, and particularly mice are preferred for use.

Animal models may incorporate a gene coding for a polypeptide of the invention or an antisense or siRNA sequence thereto that does not occur naturally in the animal, (exogenous), or does not occur at the location in which the gene is introduced, or does not occur in the same configuration as the introduced gene. Also encompassed by the animal models are animals in which endogenous genes corresponding to a nucleic acid molecule of the invention are altered, disrupted or eliminated.

Alterations in the germ line of the animals may be achieved using any known art methods. For example genes may be incorporated into the genome of an animal through microinjection of zygotes (Brinster et al., PNAS (USA) 82:4438-4442 (1985); through viral integration using retrovirus infection of blastomeres or blastocoels (Jaenuch, R; PNAS (USA) 73:1260-1264 (1976), Johner, D et al., Nature 298:623-628 (1982); or by transformation of embryonic stem cells (Lovel-Badge, R. H., Tetracarcinomas and Embryonic Stem Cells: A Practical Approach, Robertson, E. J. et al., DRL Press, Oxford, 153-182 (1987). See also Houdebine, Transgenic Animals—Generation and Use (Harwood Academic, 1997).

In another aspect, the present invention provides methods of diagnosing and/or prognosing prostate cancer, PIN or a predisposition to developing same in a patient.

In one embodiment the method is carried out by determining the expression level of a nucleic acid molecule of the invention such as PSPU 43 (SEQ ID NO:3) in a patient sample. An alteration in the expression level of the molecule compared to a control level of the molecule indicates that the subject has PIN, PRC, or is at risk of developing same. Alterations in expression levels of the molecules include identifying the presence or absence of the molecule from the patient sample.

In another embodiment the invention provides a method of testing for prostatic intraepithelial neoplasia (PIN), prostate cancer (PRC) status in a patient, the method comprising determining the expression level of PSPU 43 (SEQ ID NO:3) or other nucleic acid molecule of the invention in a patient sample, wherein an increase in expression level compared to a control level of said molecule indicates that the patient has PIN, PRC status or is at risk of developing PIN or PRC.

The expression level of a molecule of the invention can be considered to be altered, including increased, if the expression level differs from the control level by a statistically significant amount. Usually by more than 5%, more than 10%, more than 20% more than 30%, more than 40%, preferably by more than 50% or more compared to the control level. Statistically significant may alternatively be calculated as P<0.05. In a further alternative, deviation can be determined by recourse to assay reference limits or reference intervals. These can be calculated from intuitive assessment or non-parametric methods. Overall these methods calculate the 0.025, and 0.975 fractiles as 0.025*(n+1) and 0.975 (n+1). Such methods are well known in the art. See for example the Immunoassay Handbook, 3rd edition, ed. David Wild. Elsevier Ltd, 2005; and Solber H. Approved Recommendation (1987) Collected reference values. Determination of reference limits. Journal of Clinical Chemistry and Clinical Biochemistry 1987, 25:645-656.

Presence of a marker absent in a control, or absence of a marker present in a control are also contemplated as changes in expression levels.

The presence of the markers and their level of expression in the sample may be determined according to methods known in the art such as Southern Blotting, Northern Blotting, FISH or quantative PCR to quantitate the transcription of mRNA [(Thomas, Pro. NAH, Acad. Sci. USA 77: 5201-5205 1980), (Jain K K, Med Device Technol. 2004 May; 15(4):14-7)], dot blotting, (DNA analysis) or in situ hybridization using an appropriately labelled probe, based on the marker sequences provided herein.

Accordingly, the invention also provides an assay for detecting the presence of a nucleic acid molecule of the invention, preferably PSPU 43 (SEQ ID NO:3) in a sample, the method comprising:
(a) contacting the sample with a polynucleotide probe which hydridises to the nucleic acid sequence under stringent hybridisation conditions; and
(b) detecting the presence of a hybridisation complex in the sample.

Preferably the hybridisation probe is a labelled probe. Examples of labels include fluorescent, chemiluminescent, radioenzyme and biotin-avidin labels. Labelling and visualisation of labelled probes is carried out according to known art methods such as those above.

For convenience the nucleic acid probe may be immobilized on a solid support including resins (such as polyacrylamides), carbohydrates (such as sepharose), plastics (such as polycarbonate), and latex beads.

As discussed above the nucleic acid molecule probe may be an RNA or DNA molecule. Preferred probes include

Pspu43
(SEQ ID NO: 15)
Forward 5′-AACAAATATAAAGTACCAGACACTCCA -3′
(SEQ ID NO: 16)
Reverse 5′-ATCTCCAGATCTTCCTTCTAGCC -3′

The expression level of the nucleic acid marker may be determined using known art techniques such as RT-PCR and electrophoresis techniques including SDS-PAGE. Using these techniques the DNA or cDNA sequence of a nucleic acid molecule of the invention, and PSPU 43 (SEQ ID NO:3) in a patient sample is amplified, and the level of DNA or cDNA or RNA measured.

In an alternate method the DNA, cDNA or RNA level may be measured directly in the sample without amplification.

A currently preferred method is Northern blot hybridization analysis. Probes for use in Northern blot hybridization analysis may be prepared based on the marker sequences identified herein. A probe preferably includes at least 10, at least 15, at least 20, at least 30, at least 40, at least 50, preferably 75, preferably 100, or more preferably 200 or more contiguous nucleotides of a reference sequence.

Alternatively, the expression level may be measured using reverse transcription based PCR (RT-PCR) assays using primers specific for the nucleic acid sequences. If desired, comparison of the expression level of the marker in the sample can be made with reference to a control nucleic acid molecule the expression of which is independent of the parameter or condition being measured. A control nucleic acid molecule refers to a molecule in which expression does not differ between the PIN/PRC state and the healthy state. Expression levels of the control molecule can be used to normalise expression levels in the compared populations. An example of such a control molecule is transgelin 2. The markers will change expression levels with disease.

Alternatively, for peptide markers, antibodies may be employed that can recognize specific duplexes, including DNA duplexes, RNA duplexes, and DNA-RNA hybrid duplexes or DNA-protein duplexes. The antibodies in turn may be labelled and the assay may be carried out where the duplex is bound to a surface, so that when the duplex is formed on the surface the presence of the antibody bound to the duplex can be detected.

Accordingly, in another aspect the invention provides an assay for detecting the presence in a patient sample of a polypeptide encoded by a nucleic acid molecule of the invention or a functionally equivalent variant or fragment thereof, the method comprising contacting the sample with an antibody of the invention under conditions in which immunocomplexes form, and detecting the presence of bound polypeptide in the sample.

A reverse test in which antibodies of the invention are detected in the sample is also feasible. In that instance the sample is contacted with a peptide of the invention under conditions suitable for immunocomplex formation and the presence of bound antibody is detected.

Immunoassays commonly available in the art for this purpose include radioimmunoassay, (RIA), enzyme immunosorbant assays (ELISA) and the like (Lutz et al., Exp. Cell. Res. 175: 109-124 (1988).

Marker expression may alternatively be measured by immunological methods such as immunohistochemical staining of cells or tissue sections and assay of cell culture or body fluids to quantitate directly the expression level. Antibodies useful for immunohistochemical staining and/or for assay of sample fluids are preferably either monoclonal or polyclonal and are discussed in greater detail below. Conveniently the antibodies may be prepared against a polypeptide of the invention or against a synthetic peptide based on the DNA sequences disclosed herein, or against exogenous sequence fused to DNA of a nucleic acid molecule of the invention, (particularly PSPU 43) and encoding a specific antibody epitope.

Prostate health monitoring from blood cells using the biomarkers of the invention may be carried out using these techniques, for example as set out in “Cytogenetic evidence that circulating epithelial cells in patients with carcinoma are malignant” by Fehm et al Clinical Cancer Research, 8:2073-2084 (2002).

Urine sampling is also feasible. The urethra passes through the prostate leading to prostate cells being passed into urine. A prostate test for the nucleic acid PCA3 marker in urine has been developed by Bostwick Laboratories (http://bostwicklaboratories.com/patientservices/uPM3.html). Similar tests may be employed for PSPU 43.

Alteration in the expression of one or more of the PSPU markers in a patient sample compared to the normal control level indicates that the patient suffers from or is at risk of developing PIN or PRC. Whether the alteration is an increase or decrease may depend on the stage of the disease. Generally, PSPU 43 has been shown to be over-expressed in prostate cancer patients. However, under-expression for example in advanced stages of prostate cancer is also feasible.

Other markers can also be used in association with PSPU markers of the invention. Useful markers include known markers of prostate cancer such as PCA3, PSA. Transgelin 1 which has been shown to be under-expressed in prostate cancer patients may also be used. It may also be useful to include a benchmark or reference marker which does not change with disease state Transgelin 2 may be useful for this purpose. Correlating the level of the PSPU marker with other markers can increase the predictive diagnostic of monitoring value of the PSPU marker of the invention. Use of PSPU 43 with known prostate cancer markers can increase the predictive or diagnostic value of patient outcome.

Analysis of a number of peptide markers can be carried out simultaneously or separately using a single test sample. Simultaneous or two site format assays are preferred. Microassay or biochip analysis are particularly useful. The assays or chips can have a number of discreet addressable locations comprising an antibody to one or more markers including a PSPU marker of the invention. US2005/0064511 provides a description of chips and techniques useful in the present invention.

In another embodiment, the present invention therefore provides a method of monitoring response to treatment of PIN or PRC in a subject, the method comprising determining the expression level of a nucleic acid molecule of the invention, preferably PSPU 43 (SEQ ID NO:3) in a patient sample, and comparing the level of said molecule to a control level, wherein a statistically significant change in the determined level from the control level is indicative of a response to the treatment.

A statistically significant increase in the PSPU molecule, particularly PSPU 43 is indicative of PIN or PRC or the results can be correlated with changes to non-PSPU 43 markers such as including those discussed above. Changes in these marker levels from a control, coupled with an increase in the PSPU molecule compared to a control may be more indicative of PRC or PIN.

Where a subject is to be monitored, a number of biological samples may be taken over time. Serial sampling allows changes in marker levels, particular PSPU 43 to be measured over time. Sampling can provide information about onset of cancer, the severity of the cancer, which therapeutic regimes may be appropriate, response to therapeutic regimes employed, and long term prognosis. Analysis may be carried out at points of care such as in doctors offices, on clinical presentation, during hospital stays, in outpatients, or during routine health screening.

The methods of the invention may also be performed in conjunction with an analysis of one or more risk factors such as, but not limited to age, family history and ethnic background.

The methods herein can be used as a guide to therapy. For example, what therapies to initiate and when.

In a further aspect, the invention provides a kit comprising one or more detection reagents which specifically bind to a PSPU nucleic acid marker molecule of the invention or a polypeptide encoded by the nucleic acid sequence. Preferably, the kit includes PSPU43 (SEQ ID NO:3). The detection reagents may be oligonucleotide sequences complementary to a portion of the PSPU marker, could be designed to nucleic acid or peptide sequences known to flank the PSPU marker or antibodies which bind to the polypeptides encoded by the PSPU marker. The reagents may be bound to a solid matrix as discussed above or packaged with reagents for binding them to the matrix. The solid matrix or substrate may be in the form of beads, plates, tubes, dip sticks, strips or biochips. Biochips or plates with addressable locating and discreet microtitre plates are particularly useful.

Detection reagents include wash reagents and reagents capable of detecting bound antibodies (such as labelled secondary antibodies), or reagents capable of reacting with the labelled antibody.

The kit will also conveniently include a control reagent (positive and/or negative) and/or a means for detecting the nucleic acid or antibody. Instructions for use may also be included with the kit. Most usually, the kits will be formatted for assays known in the art, and more usually for PCR, Northern hybridization or Southern ELISA assays, as are known in the art.

Kits will also be formatted from using the nucleic acid molecules of the invention for use in screening procedures such as FISH that detect chromosomal rearrangements associated with disease and disease progression. The kit may additionally include detection reagents for the nucleic acid, and controls.

The kits may also include one or more additional markers for prostate cancer or controls including transgelin 1, transgelin 2, PCA 3 and PSA. In one embodiment all of the markers are included in the kit.

The kit will be comprised of one or more containers and may also include collection equipment, for example, bottles, bags (such as intravenous fluids bags), vials, syringes, and test tubes. At least one container holds a composition which is effective for diagnosing, monitoring, or treating PIN or PRC. The active agent in the composition is usually a compound, polypeptide or an antibody of the invention. In a preferred embodiment, an instruction or label on, or associated with, the container indicates that the composition is used for diagnosing, monitoring or treating PIN or PRC. Other components may include needles, diluents and buffers. Usefully, the kit may include at least one container comprising a pharmaceutically-acceptable buffer, such as phosphate-buffered saline, Ringer's solution and dextrose solution.

Antibodies used in the assays and kits may be monoclonal or polyclonal and may be prepared in any mammal. They are preferably prepared against a native peptide encoded or indicated by a PSPU nucleic acid sequence of the invention, or a synthetic peptide based on same, or may be raised against an exogenous sequence fused to a nucleic acid sequence encoding a PSPU peptide of the invention.

Antibody binding studies may be carried out using any known assay method, such as competitive binding assays, non-competitive assays, direct and indirect sandwich assays, fluoroimmunoassays, immunoradiometric assays, luminescence assays, chemiluminesence assays, enzyme linked immunofluorescent assays (ELIFA) and immunoprecipitation assays. Zola, Monoclonal Antibodies: A Manual of Techniques, pp. 147-158 (CRC Press, Inc., 1987); Harlow and Lome (1998) Antibodies, A Laboratory Manual, Cold Spring Harbour Publications, New York; U.S. Pat. No. 5,221,685; U.S. Pat. No. 5,310,687; U.S. Pat. No. 5,480,792; U.S. Pat. No. 5,525,524; U.S. Pat. No. 5,679,526; U.S. Pat. No. 5,824,799; U.S. Pat. No. 5,851,776; U.S. Pat. No. 5,885,527; U.S. Pat. No. 5,922,615; U.S. Pat. No. 5,939,272; U.S. Pat. No. 5,647,124; U.S. Pat. No. 5,985,579; U.S. Pat. No. 6,019,944; U.S. Pat. No. 6,113,855; U.S. Pat. No. 6,143,576; U.S. Pat. No. 5,955,371; U.S. Pat. No. 5,631,171 and US 2005/0064511.

For example, one type of sandwich assay is an ELISA assay, in which case the detectable moiety is an enzyme. ELISA is particularly useful for predicting, detecting or monitoring PIN or PRC.

Alternate analytical techniques useful herein include mass spectrometry analysis such as surface-enhanced laser desorption and ionization (SELDI), electrospray ionization (ESI) and matrix assisted laser-desorption ionization (MALDI).

For immunohistochemistry, the tissue sample may be fresh or frozen or may be embedded in paraffin and fixed with a preservative such as formalin, for example.

In one kit embodiment a PSPU detection reagent is immobilized on a solid matrix such as a porous strip to form at least one PSPU detection site. The measurement or detection region of the porous strip may include a plurality of detection sites, such detection sites containing a PSPU detection reagent. The sites may be arranged in a bar, cross or dot or other arrangement. A test strip may also contain sites for negative and/or positive controls. The control sites may alternatively be on a different strip. The different detection sites may contain different amounts of immobilized nucleic acids eg, a higher amount in the first detection site and lower amounts in subsequent sites. Upon the addition of a test biological sample the number of sites displaying a detectable signal provides a quantitative indication of the amount of PSPU present in the sample.

In a further aspect, the invention provides an assay comprising one or more nucleic acid sequences which bind to one or more of the PSPU nucleic acid sequences of PSPU 43. A large range of sense and antisense probes and primers can be designed from the nucleic acid sequences for the PSPUs herein. The expression level of the PSPU sequence is identified using known art techniques discussed above. The array can be a solid substrate e.g., a “chip” as described in U.S. Pat. No. 5,744,305 or a nitrocellulose membrane.

Proteins expressed by the PSPU marker herein may also be used in assays, and results compared to expression levels of the same protein expressed in a normal sample. Protein presence and quantity may be assessed using assay formats known in the art and discussed herein.

In a further aspect, the invention provides a method for screening for a compound that alters the expression of a nucleic acid molecule of the invention, particularly PSPU 43 (SEQ ID NO:3). In broad terms, a test compound is contacted with a peptide encoded by a nucleic acid molecule (marker) of the invention, the biological activity of the peptide is assessed and a compound selected that alters the biological activity of the molecule in the absence of the compound, or that binds to the peptide. In an alternate embodiment a test cell that expresses the molecule is contacted with a test compound and a compound selected that alters the expression level of the marker compared to that in the absence of the compound. Such compounds include molecules that agonize or antagonize the nucleic acid molecule expression.

More specifically, screening assays for drug candidates are designed to identify compounds that bind, preferably specifically to, or complex with the polypeptides encoded by nucleic acid molecule (marker) identified herein or a biologically active fragment thereof, or otherwise interfere with the interaction of the encoded peptides with other cellular proteins. Such screening assays include assays amenable to high-throughput screening of chemical libraries, making them particularly suitable for identifying small molecule drug candidates. Small molecules generally with a molecular weight below 500 Daltons, contemplated include synthetic organic or inorganic compounds, including peptides, preferably soluble peptides, (poly)peptide-immunoglobulin fusions, and, in particular, antibodies including, without limitation, poly and monoclonal antibodies and antibody fragments, single-chain antibodies, anti-idiotypic antibodies, and chimeric or humanized versions of such antibodies or fragments, as well as human antibodies and antibody fragments.

Test compounds of the present invention can be obtained from a wide range of known compounds, unknown compounds obtained from natural sources such as plant, extracts and microorganisms, or using any of the numerous approaches in combinatorial library methods known in the art. See for example Lam Anticancer Drug Des. 12: 1 145 (1997) and DeWitt et al. PNAS 90:6909 (1993).

The assays can be performed in a variety of formats, including protein-protein binding assays, biochemical screening assays, immunoassays and cell based assays, which are well characterized in the art. All assays are common in that they call for contacting the drug candidate with a peptide encoded by a PSPU nucleic acid molecule identified herein under conditions and for a time sufficient to allow these two components to interact.

If the candidate compound interacts with but does not bind to a particular peptide encoded by a marker identified herein, its interaction with that peptide can be assayed by methods well known for detecting protein-protein interactions. Such assays include traditional approaches, such as, cross-linking, co-immunoprecipitation, and co-purification through gradients or chromatographic columns. In addition, protein-protein interactions can be monitored by using a yeast-based genetic system, see, for example, description by Fields and co-workers [Chevray et al., PNAS 89: 5789-5793 (1991). Clontech, California, USA provides a kit (MATCHMAKER™) for identifying such protein-protein interactions between two specific proteins using a two-hybrid technique. This system can also be extended to map protein domains involved in specific protein interactions as well as to pinpoint amino acid residues that are crucial for these interactions.

To test the ability of a test compound to inhibit binding, a reaction mixture is prepared and run in the absence and in the presence of the test compound. The reaction mixture usually contains a PSPU polypeptide described herein, the test compound, and components the marker polypeptide interacts with. A positive control may also be run. The binding (complex formation) between the test compound and the component the marker polypeptide interacts with is monitored as described above. The formation of a complex in the control reaction(s) but not in the reaction mixture containing the test compound indicates that the test compound interferes with the interaction of the test compound and its reaction partner.

Using these screening assays, compounds that alter the activity of a PSPU marker preferably PSPU 43 can be identified. Compounds that activate function of the PSPU marker are agonists. Similarly, compounds that inhibit the function of the PSPU marker are antagonists. These compounds identified using the screening methods of the invention also form part of the present invention.

When the biological activity to be detected is cell proliferation, anchorage-independent growth, invasion and migration it can be detected for example, by preparing cells which express one or more PSPU peptides, culturing the cells in the presence of the test compound, and determining the speed of cell proliferation, measuring the cell cycle, and/or colony forming activity in soft agar, modified Boyden invasion assay and migration assay.

A decrease in the binding activity or biological activity of one or more peptides encoded by a PSPU nucleic acid sequence of the invention compared to a normal control level of the marker detected by the screening method indicates that the test compound is an inhibitor or antagonist of the PSPU marker. Conversely, an increase in binding activity with, or the biological activity with the PSPU marker compared to a normal control level indicates that the test compound is an enhancer or agonist of the marker.

Peptides, non-peptide compounds, synthetic micromolecular compounds and natural compounds can be used in the screening methods of the present invention

Computer modelling of agonists and antagonists to nucleic acid molecules of the invention is also possible using well known programmes such as AUTODOCK (Dunbrack et al., 1997, Folding and Design 2:R27-42) CHARMm and QUANTA programs (Polygen Corporation, Massachusetts, USA).

The present invention also provides a PIN or PRC reference expression profile. This comprises a pattern of marker expression including a nucleic acid molecule of the invention, preferably PSPU 43. Usefully, the expression profile includes one or more additional markers selected from PCA3, transgelin 1, transgelin 2, and PSA. In one embodiment the markers are PCA3 and PSA. In another embodiment all the additional markers are included. Using the expression techniques discussed above the profile can be generated and used as a point of comparison for new patient samples in the diagnosis of PIN or PRC or a predisposition to same. The profiles can also be used to monitor a course of treatment for PIN or PRC, and as a prognosis tool for a patient identified as having PIN or PRC.

Accordingly, a further aspect of the invention provides a method of treating or preventing PIN or PRC in a patient wherein a PSPU molecule of the invention is over-expressed. The method comprises altering the expression of the PSPU marker or the activity of a peptide encoded by same. Inhibition may be effected by administration of one or more compounds obtained by the screening methods above. Alternatively, expression may be inhibited by known art methods such as administration of nucleic acid that inhibits or antagonises the expression of the marker. Antisense oligonucleotides, siRNA, intracellular antibodies and, ribozymes which disrupt expression of the marker can all be used for inhibiting expression.

Antisense-oligonucleotides corresponding to a PSPU molecule herein, preferably PSPU 43 can be used to reduce the expression level of the PSPU molecule in situations where that is required. The antisense-oligonucleotides of the present invention may act by binding to polypeptides encoded by PSPU nucleic acid molecules of the invention, or DNAs or mRNAs corresponding thereto, thereby inhibiting the transcription or translation of the markers, promoting the degradation of the mRNAs, and/or inhibiting the expression of proteins encoded by the PSPU nucleic acid molecule, and finally inhibiting the function of the proteins.

The nucleic acids that inhibit one or more gene products of overexpressed genes also include small interfering RNAs (siRNA) comprising a combination of a sense strand nucleic acid and an antisense strand nucleic acid of the nucleotide sequence encoding the PSPU marker. The term “siRNA” refers to a double stranded RNA molecule which prevents translation of a target mRNA. Standard techniques of introducing siRNA of the invention into the cell can be used in the treatment or prevention of PIN or PRC, including those in which DNA is a template from which RNA is transcribed. The siRNA may be constructed such that a single transcript has both the sense and complementary antisense sequences from the target gene, e.g., a hairpin.

The method is used to suppress gene expression of a cell with up-regulated expression of a PSPU molecule of the invention. Binding of the siRNA to the PIN or PRC marker transcript in the target cell results in a reduction of PIN or PRC protein production by the cell. The length of the oligonucleotide is at least 10 nucleotides and may be as long as the naturally occurring transcript. Preferably, the oligonucleotide is less than 100, less than 75, less than 50 or less than 25 nucleotides in length. Preferably, the oligonucleotide is 19-25 nucleotides in length.

The nucleotide sequence of siRNAs may be designed using a siRNA design computer program available from the Ambion website (http://www.ambion.com/techlib/misc/siRNA_finder.html) and as described in Yuan et al., Nucleic Acids Research 2004 vol 32, W130-W134. Nucleotide sequences for the siRNA are selected by the computer program based on the following protocol:

Selection of siRNA Target Sites:
1. Beginning with the AUG start codon of transcript, scan downstream for AA dinucleotide sequences. Record the occurrence of each AA and the 3′ adjacent 19 nucleotides as potential siRNA target sites. Harborth et al. (2003) recommend against designing siRNA against the 5′ and 3′ untranslated regions (UTRs) and regions near the start codon (within 75 bases) as these may be richer in regulatory protein binding sites. Complexes of endonuclease and siRNAs designed against these regions may interfere with the binding of UTR-binding proteins and/or translation initiation complexes.
2. Compare the potential target sites to the human genome database and eliminate from consideration any target sequences with significant homology to other coding sequences. The homology search can be performed using BLAST, as described above, and which can be found on the NCBI server at: www.ncbi.nlm.nih.gov/BLAST/
3. Select qualifying target sequences for synthesis. On the Ambion website, several preferred target sequences along the length of the gene can be selected for evaluation.

The siRNAs may inhibit the expression of the PSPU molecule and therefore be useful for suppressing the biological activity of the protein. Therefore, a composition comprising the siRNA may be useful in treating or preventing PIN or PRC in which over-expression of a PSPU molecule is implicated.

The nucleic acids that inhibit one or more gene products of overexpressed genes also include ribozymes against the over-expressed markers. Ribozymes are generally RNA molecules which possess the ability to cleave other single stranded RNA in a manner analogous to DNA restriction endonucleases.

Methods for designing and constructing ribozymes are known in the art (see for example Koizumi et al. FEBS Lett. 228: 225 (1998); Kikuchi et al., NAR 19: 6751 (1992)) and ribozymes inhibiting the expression of an over-expressed PIN or PRC protein can be constructed based on the sequence information of the nucleotide sequence encoding the PIN or PRC protein according to conventional methods for producing ribozymes. Therefore, a composition comprising the ribozyme may be useful in treating or preventing PIN or PRC.

Alternatively, the function of one or more gene products of any over-expressed genes may be inhibited by administering a compound that binds to, or otherwise inhibits the function of the gene products. For example, an antibody which binds to an over-expressed marker product or products may be useful in PIN/PRC treatment as well as in diagnostic and prognostic assays.

The present invention also relates to the use of antibodies, or a fragment of the antibody. As used herein, the term “antibody” refers to an immunoglobulin molecule having a specific structure that interacts (binds) specifically with a molecule comprising the antigen used for synthesizing the antibody or with an antigen closely related to it. An antibody binds specifically to a PSPU polypeptide of the invention if it does not bind non-PSPU polypeptides. Usually, the antibody will have a binding affinity (dissociation constant (Kd) value), for the PSPU antigen of no more than 10⁻⁷M, preferably less than about 10⁻⁸M, preferably less than about 10⁻⁹M. Binding affinity may be assessed using surface plasma resonance.

An antibody that binds to a PSPU marker polypeptide herein may be in any form, such as monoclonal or polyclonal antibodies, and includes antiserum obtained by immunizing an animal such as a mouse, rat or rabbit with the polypeptide, all classes of polyclonal, monoclonal, human antibodies and humanized and intracellular antibodies produced by genetic recombination.

Furthermore, the antibody used in the method of treating or preventing PIN or PRC may be a fragment of an antibody or a modified antibody, so long as it binds to one or more of the proteins encoded by the marker genes herein. The fragment will usually comprise the antigen binding region or a complementarity determining region of sane, or both. The antibody fragment may be Fab, F(ab′)2, and Fc or Fv or single chain Fv (scFv), in which Fv fragments from H and L chains are ligated by an appropriate linker (Huston et al. Proc. Natl. Acad. Sci. USA 85: 5879-83 (1988)).

Methods for preparing antibodies are well known in the art (see for example Antibodies: A Laboratory Manual, CSH press, eds, Harlow and Lane (1988)). Most commonly used antibodies are produced by immunizing a suitable host mammal as discussed above. Fusion proteins with PSPU proteins may also be used as immunogens.

An antibody may be modified by conjugation with a variety of molecules, such as polyethylene glycol (PEG). The modified antibody can be obtained by chemically modifying an antibody. These modification methods are conventional in the field.

Alternatively, an antibody may be obtained as a chimeric antibody, between a variable region derived from nonhuman antibody and the constant region derived from human antibody, or as a humanized antibody, comprising the complementarity determining region (CDR) derived from nonhuman antibody, the frame work region (FR) derived from human antibody, and the constant region. Such antibodies can be prepared using known art methods.

In brief, methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a PSPU polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL TDM adjuvant (moriophosphoryl Lipid A, synthetic trehalose dicorynoinycolate). The immunization protocol may be selected by one skilled in the art without undue experimentation.

Intracellular antibodies are generally single chain antibodies herein they will comprise single chain antibodies which specifically bind a PSPU polypeptide. They may be used in gene therapy by incorporating the sequence encoding the antibody into a recombinant vector and administering to cells over-expressing a PSPU polypeptide to bind and inhibit its function. Methods for producing these antibodies are known in the art. (see for example Tanaka et al., Nucleic Acids Research 31(5):e23 (2003).

Monoclonal antibodies may be prepared using hybridoma methods which are also well known in the art. See for example Kohler and Milstein, Nature, 256:495 (1975). The hybridoma cells may be cultured in a suitable culture medium, alternatively, the hybridoma cells may be grown in vivo as ascites in a mammal. Preferred immortalized cell lines are murine myeloma lines, such as MPC-11 an MOPC-21 which can be obtained, for example, from the American Type Culture Collection, Virginia, USA. Immunoassays may be used to screen for immortalized cell lines which secrete the antibody of interest. Polypeptides encoded for by the PSPU markers herein or variants or fragments thereof may be used in screening.

Accordingly, also contemplated herein are hybridomas which are immortalized cell lines capable of secreting a PSPU peptide specific monoclonal antibody.

Well known means for establishing binding specificity of monoclonal antibodies produced by the hybridoma cells include immunoprecipitation, radio-linked immunoassay (RIA), enzyme-linked immunoabsorbent assay (ELISA) and Western blot. (Lutz et al., Exp. Cell. Res. 175:109-124 (1988)). Antivirus from immunised animals may similarly be screened for the presence of polyclonal antibodies.

To facilitate detection, antibodies and fragments herein may be labelled with detectable markers that allow for direct measurement of antibody binding such as fluorescent, bioluminescent, and chemiluminescent compounds, as well as radioisotopes, magnetic beads, and affinity labels (e.g biotin and avidin). Examples of labels which permit indirect measurement of binding include enzymes where the substrate may provide for a coloured fluorescent product, suitable enzymes include horseradish peroxidase, alkaline phosphatase, malate dehydrogenase and the like. Fluorochromes (e.g Texas Red, fluorescein, phycobiliproteins, and phycoerythrin) can be used with a fluorescence activated cell sorter. Labelling techniques are well known in the art.

The monoclonal antibodies secreted by the cells may be isolated or purified from the culture medium or ascites fluid by conventional immunoglobulin purification procedures such as, for example, protein A-Sepharose, hydroxyapatite chromatography, gel electrophoresis, dialysis, or affinity chromatography.

The monoclonal antibodies or fragments may also be produced by recombinant DNA means (see for example U.S. Pat. No. 4,816,567). DNA modifications such as substituting the coding sequence for human heavy and light chain constant domains in place of the homologous murine sequences (U.S. Pat. No. 4,816,567; supra) are also possible. Production of chimeric bivalent antibodies are also contemplated herein.

The antibodies may be monovalent antibodies. Methods for preparing monovalent antibodies are well known in the art.

The anti-PSPU antibodies of the invention may further comprise humanized antibodies or human antibodies. Such humanized antibodies are preferred for therapeutic use. Humanized antibodies include human immunoglobulins in which residues from a complementary determining region (CDR) of the recipient are replaced by residues from a CDR of a non-human species. The production of humanized antibodies from non-human sources such as rabbit, rat and mouse are well known. (Verhoeyen et al, Science, 239:1534-1536 (1988); Jones et al., Nature, 321:522-525 (1986); Riechmann et al., Nature 332:323-329 (1988);

Human antibodies can also be produced using various techniques known in the art, including phage display technologies (Hoogenboom and Winter, J. Mol. Biol. 227:381 (1991)); and transgenic methods, see, for example Nature Biotechnology 14, 826 (1996); and Vaughan et al, Nature Biotechnology 16:535-539 (1998).

Bispecific antibodies are monoclonal, preferably human or humanized, antibodies that have binding specificities for at least two different antigens. Contemplated herein are bispecific antibodies wherein one of the binding specificities is for the PSPU marker, the other one is for any other antigen, and preferably for a cell-surface protein or receptor or receptor subunit.

Methods for making bispecific antibodies are known in the art. See for example Milstein and Cuello, Nature, 305:537-539 (1983) and Suresh et al., Methods in Enzymology, 121:210 (1986), Brennan et al., Science 229:81 (1985).

Bispecific antibodies may bind to two different epitopes on a given PSPU polypeptide herein. Alternatively, they may bind to an anti-PSPU and epitope which binds to molecule(s) involved in cellular defence in the cells expressing the PSPU. For example, leukocyte T-cell receptor molecules, and Fc receptors for IgG. In a further alternative, the bispecific antibodies may include an epitope which binds a cytotoxic agent such as ricin A chain, saporin, or methotrexate or a radionuclide chelator, such as EOTUBE, or DOTA.

Antibodies with greater than two specificities eg trispecific antibodies are also contemplated herein.

Heteroconjugate antibodies composed of two covalently joined antibodies are also contemplated herein. These antibodies have suggested utility in targeting immune system cells to unwanted cells (U.S. Pat. No. 4,676,980). The antibodies may be generated in vitro using crosslinking techniques known in the art.

The effectiveness of the antibody may be enhanced. For example, by introducing cysteine residue(s) into the Fe region, thereby allowing interchain disulfide bond formation in this region to generate a homodimeric antibody. Homodimeric antibodies may be generated using cross-linkers known in the art such as described in Wolff et al., Cancer Research, 53: 2560-2565 (1993).

Antiidiotypic antibodies can also be used in the therapies discussed herein, to induce an immune response to cells expressing a PSPU protein. Production of these antibodies is also well known (see for example Wagner et al., Hybridoma 16:33-40 (1997)).

Antibodies of the invention may be immobilized on a solid support: suitable supports include those discussed above for the nucleic acid sequences. Binding of antibodies to a solid support can be achieved using known art techniques. See for example Handbook of Experimental Immunology, 4th Edition, Blackwell Scientific Publications, Oxford (1986). The bound antibody is useful in the assays discussed herein.

The present invention provides a method for treating or preventing, PIN or PRC in a patient in need thereof, using an antibody against a PSPU polypeptide. According to the method, a pharmaceutically effective amount of an antibody against the PIN or PRC polypeptide is administered to the patient. Administration is at a dosage sufficient to reduce the activity of the PIN or PRC polypeptide where over-expression of a PSPU molecule of the invention, particular PSPU 43 is implicated in PIN or PRC. Alternatively, an antibody binding to a cell surface marker specific for tumor cells can be used as a tool for drug delivery. Thus, for example, an antibody against a PSPU polypeptide conjugated with a cytotoxic agent (eg maytonsinoid, fluorouracil, taxol, ricin A chain, abrin A chain, diphtheria toxin, doxorubicin, methotrexate, enomycin, gelonin, radionuclides such as ¹⁸⁶Re, ²¹²Bi, p³², I¹²⁵ and ¹³¹I) may be administered at a dosage sufficient to injure or kill tumor cells. The treatment methods may involve administration of one or more antibodies. Methods for preparing immunoconjugates useful in such methods are described in Vitetta et al., Science, 238: 1098 (1987) for example.

The present invention also relates to a method of treating or preventing PIN or PRC in a patient by administering a compound that alters the expression or activity of a PSPU polypeptide of the invention. In the case of over-expression, a compound is administered that decreases the expression or activity of the PSPU polypeptide. The compound or composition may be a vaccine comprising a PSPU polypeptide of the invention or an immunologically active fragment of said polypeptide, or a polynucleotide encoding the polypeptide or the fragment thereof. Administration of the polypeptide may induce an anti-tumor immunity in a subject. The polypeptide or the immunologically active fragments thereof may also be useful as vaccines against PIN or PRC. Vaccines comprising one or more PSPU polypeptides herein are contemplated for administration, as is administration of multiple vaccines comprising a single PSPU polypeptide. Benign tumors can be treated or prevented via inducing anti-tumor immunity in a subject. In some cases the proteins or fragments thereof may be administered in a form bound to the T cell receptor (TCR) or presented on an antigen presenting cell (APC), particularly dendritic cells (DC)

In the present invention, the term PIN or PRC vaccine refers to a substance that induces anti-tumor immunity or acts via the immune system to suppress PSPU upon inoculation. In general, anti-tumor immunity includes immune responses, induction of cytotoxic lymphocytes against tumors, induction of antibodies that recognize tumors, and induction of anti-tumor cytokine production.

The induction of anti-tumor immunity can be detected by observing the immune system response in host animal against the protein. Systems for detecting responses are well known in the art.

Polypeptides that induce cytotoxic T lymphocytes against tumor cells are useful in vaccines against PIN or PRC as are the cytotoxic T lymphocytes induced. Antigen presenting cells with the ability to induce cytotoxic T lymphocyte against PIN or PRC are also useful in vaccines against PIN or PRC. Cytotoxic T lymphocyte induction can be increased using a combination of proteins/peptides of different structure. These combinations are contemplated for use in the immunotherapy methods discussed herein.

Anti-tumor immunity by a polypeptide can also be assessed by determining antibody production against tumors. If growth, proliferation or metastasis of tumor cells is suppressed by an antibody, the polypeptide used to generate the antibody clearly has the ability to induce anti-tumor immunity.

Administering a vaccine of this invention, therefore allows for treatment and/or prevention of PIN or PRC by inducing anti-tumor immunity. Therapeutic and prophylactic treatment of PIN or PRC may include any inhibition of the growth of tumor cells, and suppression of occurrence of tumor cells, alteration in levels of PIN or PRC markers in the blood, alleviation of detectable symptoms accompanying PIN or PRC, and decrease in patient mortality. Such therapeutic and preventive effects are preferably statistically significant. For example, at a significance level of 5% or more, preferably 10% or more compared to a control.

When formulating a vaccine of the invention, polypeptides having immunological activity, or a polynucleotide or vector encoding the polypeptide may be combined with an adjuvant. An adjuvant refers to a compound that enhances the immune response against the protein when administered together (or successively) with the protein having immunological activity. Examples of adjuvants include cholera toxin, salmonella toxin, and alum but are not limited thereto. Vaccines of this invention may be combined with a pharmaceutically acceptable carrier such as sterilized water, physiological saline and, phosphate buffer. Furthermore, the vaccine may contain as necessary, stabilizers, suspensions, preservatives, surfactants and the like. The vaccine may be administered systemically or locally in single or multiple administrations. The polypeptides may be conjugated with carriers such as KLH, BSA or other proteins known in the art, when being used as an immunogen. Other therapeutic compositions are discussed below.

The present invention also provides a method of treating or preventing PIN or PRC in a subject by administering a compound that alters the expression or biological activity of PSPU nucleic acid molecule or PSPU polypeptide of the invention.

In one embodiment of this method, the therapeutic compounds include polypeptide products of under-expressed markers, or a biologically active fragment thereof, a nucleic acid encoding an under-expressed gene downstream of expression control elements permitting expression of the gene in the PIN or PRC cells, compounds that increase the expression level of the marker endogenously existing in the PIN or PRC cells. These compounds can be obtained using the screening methods herein. To deliver a missing gene or protein to a cell a retrovirus system can be used. Such systems are known in the art. See for example U.S. Pat. No. 5,082,670 and “Retroviral Vectors” in DNA cloning: A Practical Approach, Volume 3, DRL Press, Washington (1987). As discussed above vectors can be incorporated into a cell by techniques such as microinjection, transfection, transduction and electroporation amongst others (Sambrook et al., supra). Gene therapy can be used to inhibit inappropriate over expression, or to enhance expression of a PSPU 43 molecule or polypeptide.

The present invention also provides compositions for treating or preventing PIN or PRC comprising pharmaceutically effective amounts of:

a compound identified by a method of the invention; or an antibody or fragment thereof that binds to a PSPU polypeptide of the invention. The compositions may include two or more of such compounds, antibodies and polypeptides or combinations thereof. Also included in the composition is a pharmaceutically acceptable carrier excipient or diluent. Also provided are pharmaceutical compositions comprising an effective amount of at least one PSPU antisense sequence, siRNA, ribozyme or polypeptide with a pharmaceutically acceptable carrier, excipient or diluent.

Therapeutic compositions containing a compound, antisense sequence, siRNA, ribozyme, polypeptide or antibody of the invention may be prepared by mixing an effective amount of the active molecule with optional pharmaceutically acceptable carriers, excipients or stabilizers (Remington's Pharmaceutical Sciences 16th edition, Osol, A. Ed. [1980]).

An effective amount as used herein refers to any of the actives including a polypeptide, antibody, small molecule, siRNA, antisense sequence, ribozyme, agonist or antagonist disclosed herein in an amount sufficient to carry out a stated purpose. A skilled worker can determine the amount empirically using routine methods. Similarly, a “pharmaceutically effective amount” or “therapeutically effective amount” refers to an amount of active disclosed herein which is effective to prevent or treat PIN or PRC (see definition of “treat”) above.

The composition may be formulated for oral administration (eg capsules, tablets, lozenges, powders, syrups, and the like), for parenteral administration (eg intravenous solutions, subcutaneous, intramuscular or suppository formulations), for topical administration (eg creams, gels), for inhalation (eg intranasal, intrapulmonary) or such other forms of administration as are known in the art.

Acceptable carriers, excipients, or stabilizers are well known in the art. They must be nontoxic to recipients at the dosages and concentrations employed, and include buffers (eg phosphoric and citratic acid), water, oils, particularly olive, sesame, coconut and mineral and vegetable oils; carbohydrates including lactose, glucose, mannose, or dextrins; chelating agents such as EDTA; sugars such as sucrose, mannitol, trehalose or sorbitol; salt-forming counter-ions such as sodium; metal complexes (e.g., Zn-protein complexes); non-ionic surfactants such as TWEEN™, or polyethylene glycol (PEG)).

For tablets, diluents such as carbonates (eg sodium and calcium) phosphates (such as calcium phosphate) or lactose are commonly used with antioxidants, granulating and disintegrating agents (eg corn starch), binding agents such as starch, and lubricating agents such as stearic acid and magnesium stearate. Tablets may be coated to facilitate ingestion, stability or disintegration.

Injectable compositions are usually prepared with wetting agents (such as polyoxyethylene stearate, lecithin, and polyoxyethylene-sorbitol monooleate) and suspending agents (such as methylcellulose, sodium alginate, and gum tragacanth) as well as diluents.

The compositions may also include additives such as colourants, antioxidants (such as ascorbic acid), sweeteners, thickening agents, (eg paraffin, beeswax), flavouring agents and preservatives (such as alkyl parabens, phenol, resorcinol and benzalkonium chloride) as appropriate.

Any conventional technologies may be employed to produce tablets, topical and intravenous formulations, syrups, oil-in-water emulsifiers, inhalants and the like (Remington's supra).

Liposomes can also be used to deliver the actives into cells. Where antibody fragments are used, the smallest inhibitory fragment which specifically binds to the binding domain of the target protein is preferred. Peptides can be chemically synthesized produced recombinantly as discussed above, or as otherwise known in the art. See for example PNAS USA 90, 7889-7893 (1993).

The therapeutic compositions may also contain one or more additional active agents. Other actives selected should not have significant adverse effects on the main active agent discussed above. Examples of additional active agents are cytotoxic agents, cytokines, chemotherapeutic agents such as Taxol® and cisplatin, and or growth inhibitory agents. The actives are present in combination in therapeutically effective amounts. The actives may be formulated as part of the therapeutic composition, or separately for simultaneous or sequential use with the therapeutic composition.

The active agent may also be formulated as in microcapsules or aqueous suspensions for example, with suspending agents such as sodium alginates, methylcelluloses (eg methylcellulose, carboxymethyl cellulose, hydroxlpopylmethyl cellulose) or in macroemulsions. Such techniques are disclosed in Remington's Pharmaceutical Sciences 16th edition, Osol, A. Ed. (1980).

The compositions to be used for in vivo administration must be sterile. This is readily accomplished by filtration through sterile filtration membranes or other known art techniques.

Sustained-release preparations may be prepared. Suitable examples of sustained-release preparations microcapsules discussed above. Examples of sustained-release matrices include polyesters, and hydrogels.

In immunoadjuvant therapy, administration of the proteins, antibodies or compounds of the instant invention may be used in conjunction with chemotherapy, chemical androgen ablation therapy, or radiation therapy or the separate, simultaneous or sequential administration of other anticancer agents. Preparation and dosing schedules for agents may be used according to manufacturers' instructions or as determined by the skilled practitioner. Preparation and dosing schedules for chemotherapeutic agents is given in for example Chemotherapy Service Ed., M. C. Perry, Williams & Wilkins, Baltimore, Md. (1992). For the treatment or reduction in the severity of PIN or PRC or its symptoms, the appropriate dosage of an active of the invention will depend on the patients age, type and severity of disease to be treated, whether the agent is administered for preventive or therapeutic purposes, previous or other concurrent therapies, the route of administration, the patient's clinical history and response to the active, according to the well known principles of medicine. The compound may be administered to the patient once only, continuously or repeatedly. For example daily, weekly, monthly, multiple times in a day, and administration may be regular, intermittent or at spaced intervals.

Depending on the type and severity of the disease, about 1 μg/kg to 15 mg/kg (e.g., 0.005 to 20 mg/kg, preferably 0.1 to 1 mg/kg) of an active of the invention including a compound, composition, nucleic acid molecule, polypeptide or antibody of the invention is an initial candidate dosage for administration to the patient, in single or divided doses or by continuous infusion. A typical daily dosage might range from about 1 μg/kg to 100 mg/kg, more usually 1 mg to 75 mg/kg, or more, depending on the factors highlighted above. Treatment may be effected until the disease or its symptoms have abated or a decision is made to terminate. The treatment regime can be monitored by using assays herein discussed or other conventional monitoring techniques.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents; or such sources of information, in any jurisdiction, are prior art, or form part of the common general knowledge in the art.

The invention will now be illustrated in a non-limiting way by reference to the following Examples:

### Example 1

### Identification of Chromosome 8, Prostate-Enriched Sequences: Pspu1, Pspu2, Pspu8 and Pspu43

### Introduction

We have developed a system of automated datamining which we refer to as Data-Panning. The starting point for this system is the capture of transcripts (mRNAs) from tissue samples and their conversion to stable products (cDNAs) in the form of cDNA libraries. The extensive sequencing of cDNA libraries has resulted in deposition of large numbers of Expressed Sequence Tags (ESTs) in GenBank. These expressed sequences are the source of the ESTs in the UniGene databases (Schuler et al. 1996). Currently, there are about 4.1 million human ESTs/mRNAs in the human UniGene database.

UniGene partitions the ESTs imported from GenBank into a non-redundant set of gene-oriented clusters, with each UniGene cluster nominally containing sequences that represent transcripts from a single gene (Schuler et al. 1996). A key feature of UniGene is the assignment of a dbEST library ID to ESTs. Since the dbEST library ID identifies the tissue from which the dbEST library was constructed, this ID is a computationally unambiguous marker of the tissue source of the EST in a UniGene cluster.

dbEST libraries are derived from a wide range of organs and tissues. If these libraries were representative of the body as a whole, the aggregate of the individual library transcriptomes would reflect a whole-body transcriptome. At the same time, individual libraries would reflect regional differences in transcriptomes attributable to organs and tissues. UniGene clusters containing a high proportion of ESTs from a single tissue would be identifiable against the overall UniGene background.

We have used this computational method to identify enriched gene expression profiles in prostate tissues. No prior knowledge of the function or likely distribution of these genes is required. Four transcripts located on Chromosome 8 were identified using our approach and are described in this work. We named these transcripts Prostate specific unigene 1 (Pspu1), Prostate specific unigene 2 (Pspu2), Prostate specific unigene 8 (Pspu8) and Prostate specific unigene 43 (Pspu43). The EST details are as follows:

TABLE 1
List of EST sequences making up
each Chromosome 8 candidate

| | GenBank | |
|---|---|---|
| | Accession | |
| Marker | Numbers | Full EST sequence |
| Pspu1 | AA635472 | |
| | AI659328 | |
| | AI659339 | |
| | BX112742 | |
| | AW014583 | |
| Pspu2 | CB050448 | |
| | BX113278 | |
| | AI420913 | |
| | AI927409 | |
| | AW293795 | |
| Pspu8 | BX283231 | |
| | CF139278 | |
| | BM043676 | |
| | BU543602 | |
| Pspu43 | AI611685 | ctttctttttttttgctctatctccagatcttc |
| | | cttctagccaaactcctttgcacccaaaaagca |
| | | gcctttgctttcttgagatgaaagaacattcat |
| | | gaaaatcatccctctactggagtcctgtagcaa |
| | | ttcctgtgatttccacttacctgactatgtaca |
| | | caagcccagatacctggcttagtgtggggacag |
| | | agcagagtgaccaagagtccagacctagagcct |
| | | gcttgcctgggttcaaatctcatctctaccact |
| | | cagtaaactctgtcccactttcctcatctgaaa |
| | | aatgggcataacaatagtcccttatctacagg |
| | | (SEQ ID NO: 28) |
| | AI418055 | ttttttttttttttgctctatctccagatcttc |
| | | cttctagccaaactcctttgcacccaaaaagca |
| | | gcctttgctttcttgagatgaaagaacattcat |
| | | gaaaatcatccctctactggagtcctctagcaa |
| | | ttcctgtgatttccacttacctgactatgtaca |
| | | caagcccagatacctggcttagtgtggggacag |
| | | agcagagtgaccaagagtccagacctagagcct |
| | | gcttgcctgggttcaaatctcatctctaccact |
| | | cagtaaactctgtcccactttcctcatctgaaa |
| | | aatgggcataacaat |
| | | (SEQ ID NO: 29) |
| | BF446403 | tttttttttttttttgctctatctccagatctt |
| | | ccttctagccaaactcctttgcacccaaaaagc |
| | | agcctttgctttcttgagatgaaagaacattca |
| | | tgaaaatcatccctctactggagtcctctagca |
| | | attcctgtgatttccacttacctgactatgtac |
| | | acaagcccagatacctggcttagtgtggggaca |
| | | gagcaaagtgaccaagagtccaaacctagagcc |
| | | tgcttgcctgggttcaaatctcatctctaccac |
| | | tcagtaaactctgtcccactttcctcatctgaa |
| | | aaatgggcataacaatagtcccttatctcaca |
| | | (SEQ ID NO: 30) |
| | BF222603 | ctttctttttttttgctctatctccagatcttc |
| | | cttctagccaaactcctttgcacccaaaaagca |
| | | tgcctttgctttctgagatgaaagaacattcat |
| | | gaaaatcatccctctactggagtcctctagcaa |
| | | ttcctgtgatttccacttacctgactatgtaca |
| | | caagcccagatacctggcttagtgtggggacag |
| | | agcagagtgaccaaqagtccagacctagagcct |
| | | gcttgcctgggttcaaatctcatctctaccact |
| | | cagtaaactctgtcccactttcctcatctgaaa |
| | | aaatgggcataacaatgtcccttatctcacagg |
| | | tttttagtaaaattaaatgagttaatttaattt |
| | | ttctaagcact |
| | | (SEQ ID NO: 31) |
| | BX109457 | tttattaacaaatataaagtaccagacactcca |
| | | agtgcttagaaaattaaattaactcatttaatt |
| | | ttactaaaaacctgtgagataagggactattgt |
| | | tatgcccatttttcagatgaggaaagtgggaca |
| | | gagtttactgagtggtagagatgagatttgaac |
| | | ccaggcaagcaggctctaggtctggactcttgg |
| | | tcactctgctctgtccccacactaagccaggta |
| | | tctgggcttgtgtacatagtcaggtaagtggaa |
| | | atcacaggaattgctagaggactccagtagagg |
| | | gatgattttcatgaatgttctttcatctcaaga |
| | | aagcaaaggctgctttttgggtgcaaaggagtt |
| | | tggctagaaggaagatctggagatagagcaaaa |
| | | aaaaagaaagaaaaaaaaaaaaaaa |
| | | (SEQ ID NO: 32) |
| | AA658380 | ttttttttttgctctatctccagatcttccttc |
| | | tagccaaactcctttgcacccaaaaagcagcct |
| | | ttgctttcttgagatgaaagaacattcatggaa |
| | | atcatccctctactggagtcctctagcaattcc |
| | | tgtgatttccacttacctgactatgtacacaag |
| | | cccagatacctggcttagtgtggggacagagca |
| | | gagtgaccaagagtccagacctagagcctgctt |
| | | gcctgggttcaaatctcatctctaccactcagt |
| | | aaactctgtcccactttcctcatctggtcgac |
| | | (SEQ ID NO: 33) |

### Systems and Methods

The data recorded in each UniGene cluster provides a method for generating enriched gene expression profiles for any tissue or cell type for which a cDNA library has been sequenced and allocated a dbEST library ID. Each set of ESTs clustered by the UniGene algorithm is allocated a UniGene number. This number heads the cluster entry in the UniGene database along with any known information about the gene from which the cluster arose, any STS markers for the gene, possible protein similarities, chromosome locations, etc. A field within the UniGene record called ‘scount’ indicates how many sequences form the UniGene cluster. The final section of each UniGene record lists all the sequences that form the cluster. The format of this list includes the accession number for each sequence and a dbEST library ID number if the EST was sequenced as part of a cDNA library. The dbEST ID number acts as a marker for the biological source of a given sequence.

Generation of a gene expression profile from this information relies on the large number of randomly sequenced cDNA libraries and the dbEST library numbering system. If a gene is expressed solely by one tissue then only libraries constructed from that tissue have representative sequence from that gene. By determining the dbEST library IDs of each UniGene cluster, tissue specific gene expression is shown where all ESTs are derived from libraries constructed only from a single tissue.

Most genes are not completely specific to one tissue but show a distribution over a range of tissues. In a randomly sequenced cDNA library, genes expressed in high abundance will be sequenced more frequently than those expressed at low levels. This will be reflected in UniGene. Sequences will be tagged with dbEST library IDs from the tissues in which the gene is highly expressed more often than from tissues where it is expressed at low levels. This means that the number of times a sequence is tagged with a dbEST ID number from a tissue of interest within a UniGene cluster could indicate the level of gene expression in that tissue. This can be expressed as a percentage of the total number of ESTs that contributed to the UniGene cluster. We have used this approach to obtain biomarkers specific to prostate.

### Algorithm

UniGene data files (Hs.data.gz) were downloaded from ftp://ftp.ncbi.nlm.nih.gov/repository/UniGene. These files were edited using three Perl scripts that utilize the Bioperl toolkit (Stajich et al. 2002). Specifically, these scripts call the Bio::Cluster::Unigene and Bio::ClusterIO modules. The first script, “lib_extract” automatically reviewed the number of EST sequence lines in each UniGene cluster and binned those UniGenes where the number of contributing sequences was above a specified threshold level.

This threshold level was set by defining the variable “$threshold” equal to 4 and comparing it with the scount of each UniGene cluster. Scount is the total number of sequences that contributed to the cluster. Any record was discarded if the number of sequences contributing to the cluster was the same or less than the threshold. Those with more than the threshold number of sequence lines were parsed into the in-house database for subsequent data panning (see below).

Scount was parsed from the raw Hs.data files and included those lines with no identifying dbEST library ID number. Since the subsequent computations were based on dbEST library IDs, some clusters were binned that appeared to have less than the stipulated threshold number of ESTs. Where this occurred, the lower number reflects the number of ESTs in the UniGene cluster with dbEST library IDs.

UniGene files downloaded from NIH are large (400 Mb for human). Subsequent computation is greatly facilitated by creating a series of edited in-house databases that retain solely a UniGene cluster number and dbEST ID for each of the contributing ESTs. This procedure, using the “lib_extract” script, reduced the human data files to 40 Mb.

Library catalogues (Hs.lib.info.gz) with some descriptors are available on the UniGene website. Further details on library construction are available from the UniGene Library Browser (http://ncbi.nlm.nih.gov/UniGene/). All UniGene libraries have dbEST library IDs.

Data panning was undertaken using the “lib_percentage” script. This takes a set of UniGene libraries specified by the investigator and then interrogates the edited in-house databases. These in-house databases no longer contain UniGene clusters with fewer than the threshold number of sequences. The script determines the number of EST sequence lines within each UniGene cluster that are derived from libraries of the specified set. These are expressed as a percentage of the total number of EST lines in the UniGene cluster.

### Implementation

The Human dbEST library list was downloaded from the website http://ftp.ncbi.nih.gov/repository/dbEST. The list was opened in the program BBedit, prostate libraries identified and an edited list produced using the GREP function. 290 libraries were identified as being constructed from prostate tissues. The dbEST libraries used for this analysis to identify human prostate specific sequences were: 689, 787, 792, 876, 910, 924, 925, 926, 928, 934, 935, 940, 994, 1016, 1017, 1053, 1054, 1055, 1333, 1410, 1498, 1654, 1655, 1668, 1670, 1671, 1672, 1673, 4267, 4268, 4711, 4712, 4713, 4714, 4715, 4716, 4717, 4718, 4719, 4720, 6013, 6014, 6015, 6016, 6017, 6018, 6019, 6020, 6021, 6022, 6023, 6024, 6025, 6026, 6027, 6028, 6029, 6030, 6031, 6032, 6033, 6034, 6035, 6036, 6037, 6038, 6039, 6040, 6041, 6042, 6043, 6044, 6045, 6046, 6047, 6048, 6049, 6050, 6051, 6052, 6053, 6054, 6055, 6056, 6057, 6058, 6059, 6060, 6061, 6062, 6063, 6064, 6065, 6066, 6067, 6068, 6069, 6070, 6071, 6072, 6073, 6074, 6075, 6076, 6077, 6078, 6079, 6080, 6081, 6082, 6083, 6084, 6085, 6086, 6087, 6088, 6089, 6090, 6091, 6092, 6093, 6094, 6095, 6096, 6097, 6098, 6099, 6100, 6101, 6102, 6103, 6104, 6105, 6106, 6107, 6308, 6309, 6310, 6311, 6312, 6313, 6314, 6315, 6316, 6317, 6318, 6319, 6320, 6321, 6322, 6323, 6324, 6325, 6326, 6327, 6328, 6329, 6330, 6331, 6332, 6333, 6334, 6335, 6336, 6337, 6338, 6339, 6340, 6341, 6342, 6343, 6344, 6345, 6346, 6347, 6348, 6349, 6350, 6351, 6352, 6353, 6354, 6355, 6356, 6357, 6358, 6359, 6360, 6361, 6362, 6363, 6364, 6365, 6366, 6367, 6368, 6369, 6370, 6371, 6372, 6373, 6374, 6375, 6376, 6377, 6378, 6379, 6380, 6381, 6382, 6383, 6384, 6385, 6386, 6387, 6388, 6389, 6390, 6391, 6392, 6393, 6394, 6395, 6396, 6397, 6398, 6399, 6400, 6401, 6402, 6601, 6602, 6603, 6763, 6831, 7180, 7181, 8480, 8481, 8482, 8483, 8484, 8485, 8486, 8487, 8488, 8489, 8490, 8491, 8492, 8493, 8494, 8495, 8496, 8497, 8498, 8499, 8500, 8501, 8502, 8503, 8504, 8505, 8506, 8507, 8508, 8509, 8510, 8511, 8512, 8513, 8514, 8515, 8585, 8834, 9134, 9135, 9136, 9137, 9138, 10161, 10549, 11034, 11037, 14129, 14130, 14131. These libraries were all constructed from either normal or diseased prostate material. Computations were undertaken on human UniGene build available 4 Mar. 2004. The results were imported into Microsoft Excel for sorting.

### Results and Discussion

Several studies have suggested that loss of gene sequences from the short arm of chromosome 8 (8p) is an early molecular event (Cher et al., 1994; Macoska et al., 1994; 1995; 2000; Haggman et al., 1997) in nearly all prostate cancers and, significantly, in prostatic intraepithelial neoplasia (PIN) which is the most likely precursor of prostate cancer (Bostwick, 1996). Three transcripts identified using the data panning algorithm at the 100%, 75% and 80% enrichment level (Table 2 below) are located on 8p. Two, in silico, showed a pattern consistent with loss of expression between normal and diseased tissues. Another transcript with an 83% enrichment for prostate expression was located on 8q. We named these transcripts Prostate specific unigene 1 (Pspu1), Prostate specific unigene 2 (Pspu2), Prostate specific unigene 8 (Pspu8) and Prostate specific unigene 43 (Pspu43). These transcripts have not previously been described. Pspu1 is located at 8p12, Pspu2 is found at 8p21, Pspu8 resides at 8p22-23 and Pspu143 is positioned at 8q23.

TABLE 2
Enrichment results for Pspu1, Pspu2, Pspu8 and Pspu43

| | | | | Total | |
|---|---|---|---|---|---|
| | | | EST | number of | |
| | | EST from | from | EST in | |
| UniGene | Pspu ID | prostate | other | UniGene | Percentage |
| Number | number | libraries | libraries | Cluster | (%) |
| 197095 | Pspu1 | 4 | 1 | 5 | 80 |
| 444680 | Pspu2 | 3 | 1 | 4 | 75 |
| 458397 | Pspu8 | 4 | 0 | 4 | 100 |
| 161160 | Pspu43 | 5 | 1 | 6 | 83 |

An in silico gene expression profile for Pspu1 and Pspu2 was determined using the meta-analysis system described by Stanton and Green (2001). Briefly, sequenced prostate cDNA libraries were downloaded from the NCBI (http://ncbi.nlm.nih.gov). Each library was placed into a category determined by the tissue from which it was made. This meant that all libraries made from PIN tissues were grouped together while all cDNA libraries from normal tissues formed another group. ESTs in each library were clustered based on UniGene. This gave a list of transcribed units falling within each category. By tallying the number of ESTs for a given UniGene in each category an in silico gene expression profile is generated indicative of the level of specific transcript expressed by each tissue type. This data was used to generate Table 3 below. Pspu8 and Pspu43 were not included as the libraries that gave rise to them were excluded from the meta-analysis due to the nature of their construction.

TABLE 3
Example of expression profiles of genes in normal prostatic epithelium
and progressive stages of prostate cancer.

| | | Prostatic | | | |
|---|---|---|---|---|---|
| | Normal | intraepithelial | Invasive | | Metastatic |
| Description | prostate | neoplasia | carcinoma | | lesion |
| Prostate specific | 1580 | 188% (0.001) | 103% | (ns) | 50% (0.001) |
| antigen | | | | | |
| Prostatic acid | 790 | 169% (0.001) | 83% | (ns) | 33% (0.001) |
| phosphatase | | | | | |
| Prostate specific | 20 | 0% (0.001) | 0% | (0.001) | 0% (0.001) |
| unigene 1 | | | | | |
| Prostate specific | 10 | 0% (0.001) | 0% | (0.001) | 0% (0.001) |

unigene 2

A normalized abundance score is given for normal prostate with levels in diseased tissues given as a percentage of normal expression. Levels of significance are given in parentheses as determined by Chi squared test of goodness of fit for 2 classes, ns = no significant difference.

Comparison with several genes whose expression is known to alter during prostate cancer progression agrees with this meta-analysis. For example, digital expression profiles indicate an increased expression of prostate specific antigen (PSA) in prostatic intraepithelial neoplasia in agreement with what is widely accepted (Table 3). Furthermore, meta-analysis shows down regulation of prostatic acid phosphatase (Table 3). Prostatic acid phosphatase was used to diagnose prostate cancer prior to the PSA test (Bostwick, 1996), and is now thought to be associated with loss of androgen responsiveness of prostate tumours (Meng et al., 2000).

Sequences for Pspu1, Pspu2, Pspu8 and Pspu43 were sampled multiple times from independent libraries thus giving confidence that they represent legitimate transcripts. Five ESTs contribute to UniGene cluster Hs. 197095, the sequence contig we refer to as Pspu1. These arose from three cDNA libraries which were NCI_CGAP_Pr28 (dbEST Library ID.1410), NCI_CGAP_Pr22 (dbEST Library ID.910) and NCI_CGAP_Sub2 (dbEST Library ID.2359). Libraries 1410 and 910 were constructed from normal prostate tissue. Library 2359 arose from a subtracted cDNA library which was set up to identify breast specific genes (Bonaldo et al. 1996).

Four ESTs contributed to UniGene Hs.444680 or Pspu2. These ESTs were identified in cDNA libraries NCI_CGAP_Pr28 (dbEST Library ID.1410), NCI_CGAP_Pr22 (dbEST Library ID.910) and NCI_CGAP_Sub4 (dbEST Library ID.2721). Two of these libraries were made from normal prostate (1410 and 910). The third library was another subtraction library (2721) set up to find prostate specific genes (Bonaldo et al. 1996). Our data panning algorithm was not implemented to identify subtraction library 2721 as being constructed from prostate and thus the enrichment level given to this cluster was only 75%. In fact this UniGene may reflect a transcript solely restricted to the prostate.

Pspu8 consisted of four EST sequences taken from the three clones that comprise UniGene Hs.458397. These clones were isolated from two libraries both of which were constructed from prostate carcinoma cell lines. These libraries were dbEST library 14129 and library 8834.

Six ESTs made up UniGene Hs.161160. The contig formed from these EST sequences is referred to as Pspu43. These ESTs arise from five clones found in two cDNA libraries. These libraries were NCI_CGAP_Pr28 (dbEST library ID.1410) and NCI_CGAP_Pr2 (dbEST library ID. 574). The data-panning algorithm indicated that this transcript was only 83% enriched in the prostate. Library 574, however, was not incorporated into the list of prostate specific libraries and so ESTs from this library were not tagged as being of prostate origin. Like Pspu2 this transcript is likely to be solely of prostate origin.

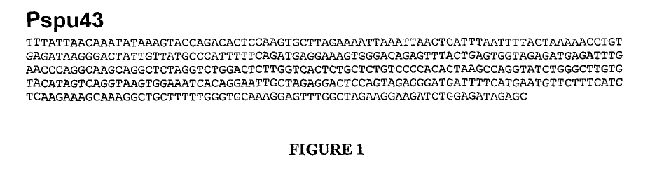

The ESTs making up Pspu1, Pspu2, Psp148 and Pspu43 were aligned to give the best consensus sequence for each candidate. The consensus sequences are given in FIG. 1 and BLASTN (Altschul et al. 1990) results against the non-redundant GenBank database at the NCBI are given in Table 4 below.

TABLE 4
Summary of BLASTN analysis for Pspu1, Pspu2, Pspu8 and
Pspu43 prostate specific candidates

| | Contig Base Pairs | Accession | |
|---|---|---|---|
| | that align to | number for best | |
| Contig | GenBank | GenBank | |
| Sequence | Candidate | alignment | Comments |
| Pspu1 | 1-353 | AC044849.12 | Genomic DNA |
| Pspu2 | 1-379 | AC090786.6 | Genomic DNA |
| Pspu8 | 1-1320 | NM_0540281 | cDNA |
| Pspu43 | 1-392 | AP001207/AP000426 | Genomic DNA |

Pspu1 and Pspu2 consensus sequences are 368 bp and 394 bp respectively in length, however, they both terminate in polyA stretches that could in reality be of variable length (18 bp and 15 bp respectively). Both sequences map on to the human genome but do not generate high scoring matches to known expressed genes. The Pspu43 consensus sequence is 392 bp long and also maps to the human genome but not expressed sequences. Pspu8 is 1320 bp long and maps to the expressed sequence for human Acyl-malonyl condensing enzyme.

### Conclusion

We have identified four nucleic acid sequences, Pspu1, Pspu2, Pspu8 and Pspu43, specific to the prostate using a UniGene data mining algorithm. Pspu1, Pspu2 and Pspu8 map to 8p12/21 border, 8p21 and 8p22-p23 respectively. Deletions from these loci are known early events in prostate cancer (MacGrogan et al. 1994). Loss of two of these sequences in disease is supported by a meta-analysis of gene expression between normal prostate and prostate cancer. Pspu43 maps to the long arm of chromosome 8 at 8q23. An adjacent region, 8q24, has recently been genetically linked to prostate cancer susceptibility (Amundadottir et al. 2006). We suggest that Pspu1, Pspu2, Pspu8 and Pspu43 would be useful markers of chromosome 8 alterations that occur as early events in the development of prostate cancer.

### Example 2

### Pspu43

### Characterization of a Prostate Disease Marker on the Long Arm of Chromosome 8

### Introduction

Pspu43 was identified as being of significance to the prostate by datamining cDNA tissue libraries using the data-panning algorithm described in Example 1 above. In total, four sequences were identified that mapped to chromosome 8 using the data-panning approach. Three were located on the short arm of chromosome 8 while Pspu43 was located on the long arm of this chromosome. Pspu43 lies close to a region on chromosome 8 that is often altered in prostrate tumours and has been linked to a genetic susceptibility to prostate cancer (Amundadottir et al., 2006).

This example summarizes our findings for Pspu43, including confirmation of genomic sequence, expression profile in a cell culture system and tissue specificity data.

### Systems and Methods

### PCR Primer Design

PCR primers for Pspu43 were designed from a contig generated by EST cluster Hs.161160. The contig was BLASTN (Altschul et al., 1990) analyzed to ensure no cloning vector sequence was incorporated in the contig. This edited sequence was loaded into a PCR primer design program (Primer3, Rozen and Skaletsky, 2000). Optimal primer pairs that generated the longest amplicon were selected and compared to the non-redundant gene sequence database at NCBI using BLASTN. Simulated PCR was performed using the AMPLIFY program (William Engles, Genetics Department, University of Wisconsin) with contig and primer sequences to ensure fidelity of match, avoid primer dimer formation and to test for possible primer secondary structures. Primers were synthesized by Invitrogen (USA). Primer sequences are given in Table 5.

TABLE 5
PCR primer sequences

| | | |
|---|---|---|
| Pspu43 | Forward | 5′-AACAAATATAAAGTACCAGACACTCCA-3′ |
| | Reverse | 5′-ATCTCCAGATCTTCCTTCTAGCC-3′ |
| Trans- | Forward | 5′-CTTCCAGAACTGGCTCAAGG-3′ |
| gelin 2 | Reverse | 5′-GAGAAGAGCCCATCATCTCG-3′ |
| PSA | Forward | 5′-CACTGCATCAGGAACAAAAGCGT-3′ |
| | Reverse | 5′-CATCACCTGGCCTGAGGAATC-3′ |

### Extraction and Amplification of RNA and Genomic DNA.

Normal prostate epithelial and stromal cells (PrEC and PrSC; Clonetics, San Diego Calif.) were grown and maintained in dedicated media (PrEGM BulletKit; Clonetics, San Diego Calif.) whilst the prostate cancer cell lines LNCaP (ATCC CRL 1740, Manassas, Va.), DU145 (ATCC HTB-81, Manassas, Va.) and RWPE-2 (ATCC CRL-11610, Manassas, Va.) were grown and maintained in RPMI 1640 medium supplemented with 10% fetal bovine serum.

RWPE-2 is a derivative of a human papilloma virus immortalized prostate epithelial cell line (RWPE-1) transformed by v-Ki-ras. It is androgen responsive, invasive and tumorogenic (Bello et al. 1997; Webber et al. 1997a). LNCaP is a derivative of a metastasized prostatic carcinoma lesion, which is responsive to androgen (Webber et al., 1997b). DU-145 is derived from a metastatic prostatic carcinoma lesion, which is unresponsive to androgen, highly invasive and tumorogenic (Webber et al., 1997b).

PrEC and LNCaP cells were seeded at a density of either 2500 cells.cm⁻² or 4000 cells.cm² respectively and cultured to 70% confluence in a humidified atmosphere of 5% CO₂ at 37° C. in 25 cm² vented flasks. Cells were harvested by trypsinisation, washed in trypsin free media and centrifuged at 500 g. Genomic DNA (gDNA) and RNA was extracted from cell pellets using TriZol (Invitrogen, Carlsbad, USA) according to the manufacturer's protocol. RNA was converted to cDNA using Superscript II (Invitrogen, Carlsbad, USA) as per manufacturer's instructions. PCR amplification was performed on 160 ng genomic DNA or 37.5 ng cDNA using the primers described above. PCR was carried out using Amplitaq Gold™ master mix (Applied Biosystems, NJ, USA). PCR conditions were optimized and established an effective annealing temperature of 65° C. Samples from all prostate cell lines were compared.

RNA from a range of tissues was purchased from Clontech laboratories Inc. (Mountain View, Calif., USA) or were donated from other research programs. These samples originated from Mammary Gland, Ovary, Testis, Kidney and Blood. These were converted to cDNA as above and used at a concentration of 37.5 ng RNA equivalent for PCR assay.

### Sequencing of PCR Products

PCR products were gel purified using the QIAgen PCR purification system (QIAGEN GmbH, Hilden, Germany). DNA was removed from 1% agarose gel using the QX1 buffer, according to the manufacturer's instructions. DNA was eluted from the purification column in sterile milliQ water.

The purified PCR amplicon was sequenced with both forward and reverse PCR primers (10 μM) using BigDye Terminator v3.1 chemistry.

### Results

Table 6 shows PCR results from both genomic DNA and cDNA synthesized from three prostate cell lines. These were LNCaP, PrEC and PrSC cell lines. PrEC and PrSC are derived from normal prostate epithelium and stromal cells, respectively. LNCaP is derived from a lymph node metastatic lesion from a patient with prostate cancer. Pspu43 sequence was detected in genomic DNA isolated from all three cell lines. This indicates that Pspu43 is part of the human genome and is not lost completely from the LNCaP cell line.

TABLE 6
Summarized PCR assay results from genomic and cDNA
isolated from PrEC, PrSC and LNCaP cell lines.

| | PrEC | PrSC | LNCaP |
|---|---|---|---|
| Genomic DNA | + | + | + |
| cDNA | + | − | + |

+ = expected PCR product/− = no PCR product

Gene expression results were obtained using RT-PCR from cDNA templates synthesized from each of the prostate cell lines. Results for all five cell lines are summarized in Table 7. RT-PCR was repeated a minimum of three times on at least two templates. Pspu43 was expressed in four of the five cell lines. That is, it is present in PrEC, LNCaP, DU145 and RWPE2 samples but not in the PrSC sample. RT-PCR results for Transgelin 2 and PSA are included for comparison.

TABLE 7
RT-PCR results from Cell Lines

| Cell Line | PSPU 43 | Transgelin 2 | PSA |
|---|---|---|---|
| PrSC | − | + | + |
| PrEC | + | + | − |
| LNCaP | + | + | + |
| DU145 | + | + | + |
| RWPE2 | + | + | + |

+ = positive for PCR test,
− = negative for PCR test

Tissue specificity for Pspu43 was examined using RT-PCR on a number of different RNA samples isolated from Ovary, Kidney, Mammary Gland, Testis and Blood. Pspu43 sequence was detected in both the Mammary Gland and Kidney but not the other tissues tested (Table 8).

TABLE 8
Summarized PCR assay results from RNA
isolated from five tissues.

| | | Mammary | | |
|---|---|---|---|---|
| Ovary | Testes | Gland | Kidney | Blood |
| − | − | + | + | − |

+ = expected PCR product/− = no PCR product

Since first identifying Hs.161160 as a UniGene cluster of interest to prostate biology it has been grouped with 142 ESTs, 7 mRNA sequences and named TFCP2L3 (Grainyhead-like 2 (Drosophila)). The original 6 EST that made up contig Pspu43 still reside in this UniGene cluster. However, contig Pspu43 does not map to any of the mRNA sequences currently associated with TFCP2L3. This was shown using 2-way BLAST between contig Pspu43 and all 7 mRNA sequences. A BLAST alignment of the Pspu43 contig to the non-redundant sequence database showed a complete match to two genomic clones only: AP000426 and AP001207. These clones are large non-annotated DNA sequences of 239,116 AND 153,936 nucleic acids respectively.

TFCP2L3 (Grainyhead-like 2 (Drosophila)) ESTs are represented highly in the prostate (expression profiler—NCBI) and by Northern Blotting (Peters et al., 2002). TFCP2L3 does not appear to incorporate the Pspu43 sequence, however. TFCP2L3 is a transcription factor that has been associated with a mutation leading to hearing loss (Peters et al. 2002).

### Conclusion

Pspu43 is an expressed RNA sequence identified as exclusively present in cDNA libraries made from both normal and cancerous prostate tissues. It is likely to be normally expressed in the prostate epithelium. Pspu43 expression is therefore a possible marker of prostate health.

### Example 3

### Urine Analysis

### Introduction

We wished to test if Pspu43 was detectable in the urine of men undergoing clinical examination for prostate disease. Urine samples were collected from the Department of Urology, Dunedin Hospital, Dunedin, New Zealand. RNA was extracted from these urine samples and subjected to RT-PCR to detect Pspu43, Transgelin 2 and PSA. These assays were scored. Patient diagnosis was made available only after RT-PCR results were obtained.

### Methods

All participants in this study gave written consent and the project received ethical approval from the Lower South Regional Ethics Committee (“Development of non-invasive, diagnostic and prognostic tests of prostate cancer” LRS/05/05/016). Men underwent prostate manipulation as part of the usual examination procedure to determine the physical state of their prostate. Prostate manipulation involved digital palpation of right and left lobes and the apex to base by sweeping the index finger three times, each side. Following this a 20 to 30 ml urine sample was collected. An equal volume of phosphate buffer (pH7.0) was added to the urine sample. This sample was stored overnight at 4° C. Cells were harvested by centrifugion at 2500 g for 15 minutes at 4° C., the supernatant removed and the cell pellet resuspended in 800 μl TriZol (Invitrogen, Carlsbad, USA). Glycogen (Invitrogen, Carlsbad, USA) was added to give a final concentration of 250 μg/ml. RNA was extracted according to the manufacturer's protocol. The RNA pellet was resuspended in 16.5 μl H₂O. Eight microlitres of the sample was treated with Dnase I (Roche, Switzerland) as per manufacture's instructions. Half of the Dnase I treated sample was converted to cDNA using Superscript II (Invitrogen, Carlsbad, USA) as per manufacturer's instructions. PCR amplification was performed on between 1 to 2.5 μl cDNA and an equivalent volume of Dnase I treated RNA using the primers described below (Table 9). PCR was carried out using Amplitaq Gold™ master mix (Applied Biosystems, NJ, USA). PCR conditions were optimized and established an effective annealing temperature of 65° C.

TABLE 9
PCR primers for Pspu43, Transgelin 2 and PSA

| | | |
|---|---|---|
| Pspu43 | Forward | 5′-AACAAATATAAAGTACCAGACACTCCA-3′ |
| | Reverse | 5′-ATCTCCAGATCTTCCTTCTAGCC-3′ |
| Trans- | Foward | 5′-CTTCCAGAACTGGCTCAAGG-3′ |
| gelin 2 | Reverse | 5′-GAGAAGAGCCCATCATCTCG-3′ |
| PSA | Forward | 5′-CACTGCATCAGGAACAAAAGCGT-3′ |
| | Reverse | 5′-CATCACCTGGCCTGAGGAATC-3′ |

### Results and Discussion

We obtained reliable RT-PCR results from urine samples provided by 8 men undergoing prostate examination for suspected disease, and one urine sample from a man who had no symptoms of prostate disease. PCR results were considered reliable if the RNA-only samples did not produce a PCR product. The enzymes used in our PCR system cannot use RNA as a template. Therefore PCR products arising from RNA-only reactions indicate the presence of genomic DNA in the sample. When this is the case it is not possible to distinguish gene expression from genomic contamination. These results are summarized in Table 10.

TABLE 10
Urine samples from 9 men

| Sample ID | PSPU 43 | Transgelin 2 | PSA | Gleason Grade |
|---|---|---|---|---|
| U “S” | − | + | + | NORMAL |
| U1 | − | + | + | No tumour |
| U3 | | | + | 7 |
| U6 | + | + | + | 6 |
| U7 | + | + | + | No tumour |
| U12 | − | + (+RT−) | + | 7 |
| U14 | + | + | + | Benign Prostatic |
| | | | | Hyperplasia |
| U20 | − | + | + | Benign Prostatic |
| | | | | Hyperplasia |
| U22 | + | + | + | 7 |

+ = positive for PCR test,
− = negative for PCR test
No reliable results for cell with no entry.

These experiments used a non-quantitative RT-PCR assay and no long term follow-up data on these patients was available. Sample U “S” is from a male with no apparent disease. No attempt was made to characterize cell populations in these urine samples. It proved challenging to extract consistent high quality RNA from urine samples and the quantity of RNA obtained was variable. As a result many samples were lost to experimental variables arising from establishing the technology.

It is clear from these results that transcripts of Pspu43, Transgelin 2 and PSA can be detected in urine. Pspu43 was detected in two of the known cancer patients (U6 & U22), one no tumour (U7) and one benign (U14). However, we have no long term follow up data on these patients. If Pspu43 is an early indicator of disease progression it is possible that patients U7 and U14 have been misdiagnosed and are in the very early stages of disease. Pspu43 was not detected in the normal (U “S”), no tumour (U1) or benign (U20) samples and it was not detected in one cancer patient (U12).

Transgelin 2 was detected in all samples, though the reading for U12 was suspect as a product was also produced from the no RT control. This was also the only known tumour sample that did not give a positive response for Pspu43. Three attempts were made to amplify two unrelated products from this sample and each time inconsistent results arose (data not shown). The PSA assay was attempted once only. The most favourable interpretation of results produced from this sample is that the RT-PCR reaction was unreliable, due either to poor quality or low concentration of RNA isolated from this sample.

PSA was detected in all samples regardless of disease state. This would indicate that PSA presence or absence from a urine sample is unlikely to be diagnostic given that it was detected regardless of prostate health or pathology of the individual.

### Conclusion

Pspu43 could be detected in the urine of men. It was detected more often in patients that were subsequently diagnosed from prostate biopsy to have prostate tumours than in men without tumours or with benign prostatic hyperplasia (BPH). PSA and Transgelin 2 were detected in all samples. Therefore, for a simple diagnostic test looking for the presence or absence of a marker neither PSA or Trangelin 2 would be suitable. Pspu43, on the other hand, may be able to be detected in patients with tumours. This supports the use of Pspu43 as a marker for prostate cancer. A problem with raised PSA as a predictor of prostate cancer progression is that it cannot distinguish prostate cancer from other pathologies. BPH and prostatitis can both raise blood PSA scores. As Pspu43 may differentiate between BPH and prostate cancer it potentially has greater sensitivity as a marker of cancer presence.

### Example 4

### Pspu43 Expression in Prostate Tumour Tissue

### Introduction

The purpose of this experiment was to examine changes in Pspu43 expression in the prostate with disease state.

Ten matched pairs of normal and lesion biopsies from single individuals were used in this study. These samples were collected from men undergoing prostatectomy for prostate adenocarcinoma and were all of Gleason Grade 6 and above. RNA was extracted from these samples and subjected to Quantitative PCR (qPCR) to determine both if Pspu43 was expressed in the prostate and to compare the relative level of expression in tumour versus normal tissue. Matched biopsies ensured that underlying genetic variation between different individuals did not confound the results. We used Transgelin 1, shown to be downregulated in other cancers (Chang et al., 2001, Shields et al., 2002) as a comparison for Pspu43 expression.

### Methods

### Tissues

Tissue biopsies were obtained from the Cancer Society Tissue Bank (Christchurch, New Zealand). Written consent was obtained from all patients donating material to the tissue bank and specific approval for this project was obtained from the Cancer Society Tissue Bank Governing Board and The Lower South Regional Ethics Committee (“Development of non-invasive, diagnostic and prognostic tests of prostate cancer” LRS/05/05/016). Tissues were supplied as frozen tissue blocks that had been snap frozen in liquid nitrogen within 15 to 30 minutes after removal from the patient. Tissue and corresponding patient details are given in Tables 11 and 12. All tumours were of histological type prostatic adenocarcinoma and all displayed perineural invasion.

TABLE 11
Tissue and Patient Details

| | | | | Max | | | | |
|---|---|---|---|---|---|---|---|---|
| | | | Glea- | Size | % vol | Lymph/ | | |
| | | | son | Tumour | of | vascular | Ne- | Mets in |
| ID | Age | N or T | grade | mm | tumour | invasion | crosis | Nodes* |
| 5 F6 | 60 | Normal | | | | No | | |
| 5 F8 | 60 | Tumour | 6 | | 25 | No | | |
| 9 B3 | 70 | Normal | | | | | | |
| 9 B4 | 70 | Tumour | 6 | | 30 | | | |
| 9 F5 | 70 | Normal | | | | | | |
| 9 F6 | 70 | Tumour | 6 | | 25 | Yes | | |
| 14 B9 | 71 | Normal | | | | | | 1/5 |
| 14 C1 | 71 | Tumour | 9 | | 85 | Yes | | 1/5 |
| 14 D1 | 49 | Normal | | | | | | |
| 14 D2 | 49 | Tumour | 7 | | 80 | +/− | | |
| 37 i9 | 67 | Normal | | | | | No | 0/12 |
| 38 A1 | 67 | Tumour | 7 | | 20 | No | No | 0/12 |
| 38 A3 | 63 | Normal | | 20 | | | Yes | 0/5 |
| 38 A4 | 63 | Tumour | 8 | 20 | 10 | Yes | Yes | 0/5 |
| 38 F1 | 66 | Normal | | | | | No | 0/14 |
| 38 F2 | 66 | Tumour | 7 | | 30 | No | No | 0/14 |
| 42 B1 | 63 | Normal | | 20 | | | No | 0/11 |
| 42 B2 | 63 | Tumour | 7 | 20 | | No | No | 0/11 |
| 43 C3 | 62 | Normal | | 25 | | | No | 0/2 |
| 43 C4 | 62 | Tumour | 7 | 25 | 20 | +/− | No | 0/2 |

*Metastases in Lymph Nodes

TABLE 12
Pathology Details

| ID | Pathology details |
|---|---|
| 5 F6/5 F8 | well-moderately differentiated prostatic adenocarcinoma |
| | arising in the left lobe adjacent to where the fresh material |
| | was taken for the tissue bank. Gleason grade 2 + 4 |
| | (score = 6) |
| 9 B3/9 B4 | Level 1 capsular invasion, Margin negative. The tumour |
| | involves approximately 30% of the gland volume and |
| | involves both the left and the right lobes with a periurethral |
| | distribution on the right side. |
| 9 F5/9 F6 | Prostatic adenocarcinoma, Gleason score 6, Level 2 capsular |
| | invasion, seminal vesicle involvement. Tumour involves |
| | 25% gland volume. Lymphovascular invasion present |
| 14 B9/14 C1 | Present at excision margins |
| 14 D1/14 D2 | level 2 capsular spread. Most of both lobes are infiltrated |
| | by tumour |
| 37 i9/38 A1 | No description |
| 38 A3/38 A4 | non-confined level 3 focal |
| 38 F1/38 F2 | confined level 2 |
| 42 B1/42 B2 | level 2, confined |
| 43 C3/43 C4 | Non-confined, level 3 established. Carcinoma is partly |
| | papillary. Areas suggestive of but not DIAGNOSTIC of |
| | vascular invasion. PSA = 21 |

### RNA Isolation

Each of the prostate tissue blocks were mounted in Cryomoulds in OCT (Lab Tek products, Tennessee, USA). Tissue was then sectioned for RNA preparation. The first and last section taken consisted of an 8 μm section which was mounted onto a slide. This slide was stored at −80° C. and used as an histology reference, if needed. Between the first and last section ten 60 μm sections were cut and placed into a pottle.

Four millilitres of TriZol (Invitrogen, Carlsbad, USA) was added to the pottle and the sections immediately homogenised for 30 seconds. The homogenate was transferred to a 15 ml falcon tube and 1.5 ml of chloroform added. The homogenate was vortexed for 15 seconds and place immediately on ice. These homogenates were then centrifuged at 4000 rpm for 15 minutes at 4° C. The top phase was removed to a clean tube and then re-extracted with 1 ml of chloroform, repeating centrifugation at 400 rpm for 15 minutes at 4° C. The top phase was again collected and transferred to a new tube. 0.53 volumes of 100% ethanol was added to the sample while vortexing vigorously. The entire nucleic acid/ethanol mix was then transferred to an Rneasy column (Qiagen, Germany) coupled to a vacuum manifold and the vacuum applied. The column, with bound nucleic acid, was then washed with 700 μl RW1 wash buffer, followed again by application of vacuum. 500 μl of wash buffer RPE was then drawn through the column under vacuum. The column was disassembled and then dried by centrifuging at 800 rpm for 15 to 30 seconds. The column was placed into a new 1.5 ml centrifuge tube, 30 μl of water added to the column and then centrifuged at 8000 rpm for a further 15 to 30 seconds. The 30 μl flow through volume was added back to the top of the column and the unit centrifuged again at 8000 rpm for 15 to 30 seconds. This column eluant contained the RNA extracted from each set of ten 60 μm sections obtained from each tissue block. The quality of the recovered RNA was tested by determining the optical density and 260/280 ratio using a Nanodrop spectrometer and also by electrophoresis using the Experion Bio-analyzer chip system (Bio-Rad, California, USA). RNA was stored at −80° C. until used.

cDNA Synthesis

One microlitre of 5 μg/μl Random Hexamers (Roche, Switzerland) was added to 600 ng of RNA. This mix was heated to 95° C. for five minutes followed by five minutes at 25° C. Samples were then transferred to ice. A cocktail of 4 μl First strand buffer (Invitrogen, Carlsbad, USA), 4 μl dNTP at 2.5 mM each, 2 μl 0.1M DTT, 0.5 μl 40 U/μl Rnase inhibitor (Invitrogen, Carlsbad, USA) and 1 μl 200 U/μl Superscript II (Invitrogen, Carlsbad, USA) was added to the RNA and mixed by pipetting. This was incubated at 42° C. for 120 minutes followed by a 10 minute incubation at 70° C. and a 1 minute incubation at 80° C.

The cDNA was cleaned using a Qiagen PCR cleanup column (Qiagen, Germany). Eighty microlitres of water and 500 μl of PB buffer were added to the 20 μl cDNA synthesis reaction. This was centrifuged through a Qiagen cleanup column at 12000 rpm for 1 minute. The flow through was discarded and the column, containing the bound cDNA was washed by centrifuging 750 μl PE buffer at 12000 rpm for 1 minute. The column was then dried by centrifugation at 12000 rpm for 1 minute. The column was transferred to a new eluant collector and 40 μl of water added. cDNA was eluted from the column by centrifuging at 800 rpm for 1 minute. A second 40 μl aliquot was added to the column and the centrifugation step repeated. The clean, eluted cDNA sample was stored at −80° C. until used.

qPCR

cDNA was diluted to a concentration of 7.5 ng/μl and 2×5 μl aliquots of each sample were pipetted into the wells of a 96 opti-well plate. RNA samples were also diluted to 7.5 ng/μl and one 5 μl aliquot pipetted into a well on a 96 well plate. No template controls to check for PCR contamination and replicate standard curve cDNA was added to each 96 well plate. To each sample was added appropriate probe/primer mixes or SYBR green (Applied Biosystems, Foster City, Calif., USA) and qPCR master mix (Applied Biosystems, Foster City, Calif., USA) was added. The plates were sealed, mixed and then briefly centrifuged to ensure contents were collected at the base of each well. qPCR was performed on an ABI7000 machine (Applied Biosystems, Foster City, Calif., USA).

Primer and probe sets are given in Table 13. Two systems were used to measure RNA transcript levels. Where single products were detected by dissociation analysis SYBR green was used as the non-specific inter-chelating dye to detect DNA amplification. In the presence of multiple bands in the dissociation analysis, a dual-labeled Taqman probe was used to provide amplicon discrimination. Each system was used in the 96 well format according to the manufacturer's protocol. Results were analysed using the SDS software package (version 1.2.3) from Applied Biosystems, California, USA.

TABLE 13
Primers and Probes used for qPCR

| Target | Forward | Probe | Reverse |
|---|---|---|---|
| Pspu43 | 5′GGCTCTAGGTC | (5′FAM)TGCTCTGTCCCCACA | 5′CCTGACTATGTA |
| | TGGACTCTTGGT3′ | CTAAGCCAGG(3′DABCYL) | CACAAGCCCAGAT3′ |
| Pspu8 | 5′GGCTGGGCCTG | (5′FAM)CTCAACGTCCTCAGC | 5′GCGGAGCATACG |
| | CTTCTGT3′ | ATTGGATGTGC(3′DABCYL) | GTGGAA3′ |
| Pspu2 | 5′CCCTGTATGAA | (5′FAM)CGGACATGAAAGGA | 5′CTATCGTTTATA |
| | ATACTAAGAGGAG | CACTAGACAAATCCACA | TTTGCCTATGTAG |
| | TCCTT3′ | (3′DABCYL) | TTACTTCAC3′ |
| Pspu1 | 5′TGGCTGTTACC | (5′FAM)AGCTATCTTGCCACTG | 5′CAGGAGGGCTGA |
| | TGCTCTTTCAC3′ | CAGACTCAGCAGT(3′DABCYL | GGTACTGTGT3′ |
| Transgelin1 | 5′AAGAATGATG | SYBR Green | 5′ACTGATGATCTG |
| (T1) | GGCACTACCG 3′ | | CCGAGGTC3′ |
| Transgelin2 | 5′CTTCCAGAACT | SYBR Green | 5′GAGAAGAGCCC |
| (T2) | GGCTCAAGG3′ | | ATCATCTCG3′ |

### Results and Discussion

RNA was extracted from each pair of matched tumour and normal samples from prostates taken from individuals undergoing prostatectomy. The average quantity of RNA obtained from each extract was 586 ng/μl with 260/280 ratios of between 1.77 and 2.

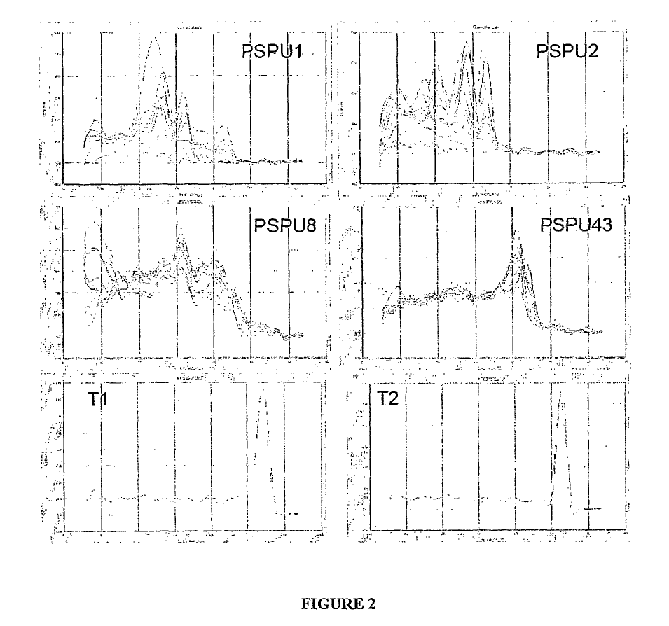

qPCR reactions for each primer/probe combination were initially optimised using cDNA from the PC3 prostate cancer cell line (ATCC CRL 1435, Virginia, USA). Assays using SYBR green technology worked well for T1 and T2 but not for any of the Pspu transcript assays, as determined from dissociation peak analysis (FIG. 2). Therefore, primer/probe sets were designed for the Pspu candidates to be used with the TaqMan qPCR system.

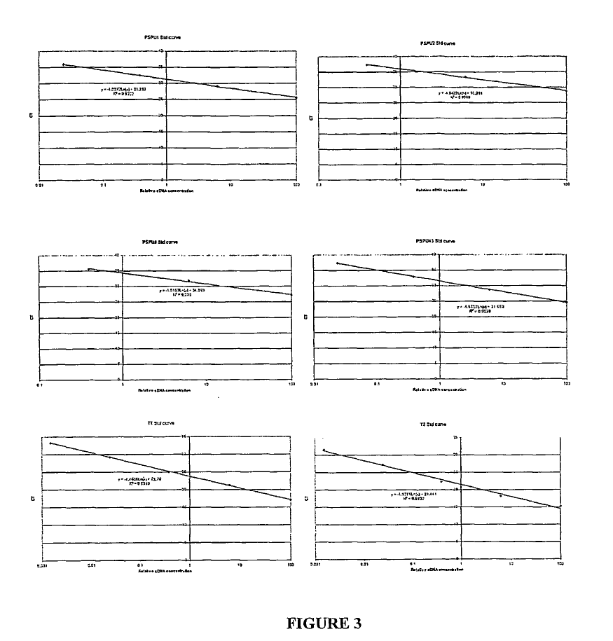

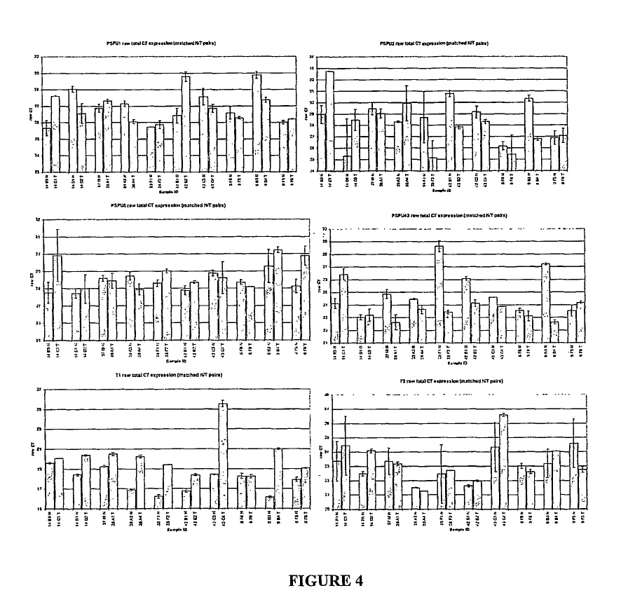

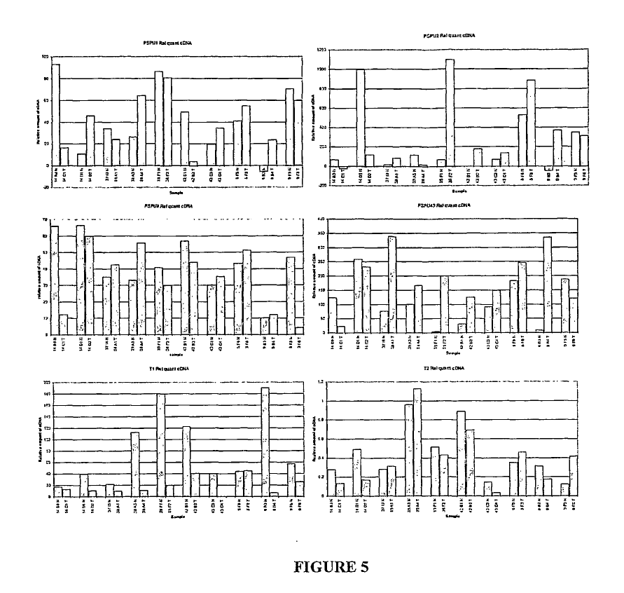

The absolute quantitation method was used to determine transcript quantity in each sample. Standard curves, generated from a universal standard of multiple stable cell lines, for each primer/probe set displayed R² values of between 0.9998 and 0.9932 (FIG. 3). Raw total CT values for each sample pair are given in FIG. 4. The standard deviation of the CT was calculated from duplicate qPCR reactions and is given as +/−1 standard deviation for each sample in FIG. 4. The relative concentration of transcript cDNA was then calculated for the average CT and calculating the corresponding cDNA value from the standard curve. This value was then corrected for genomic DNA contamination by determining the relative cDNA concentration determined from RNA-only qPCR reactions and subtracting this from the values obtained from qPCR using transcribed cDNA. The corrected relative cDNA quantification for each marker from each tissue pair is given in FIG. 5. A summary of the data is given in Table 14 along with the Gleason Grade of each patient's tumour.

TABLE 14
Prostate Tumour samples: Matched paired samples of normal
and diseased tissue from ten individuals (qPCR)

| Patient | T1 | T2 | Pspu1 | Pspu2 | Pspu8 | Pspu43 | Gleason |
|---|---|---|---|---|---|---|---|
| 5 F6/5 F8 | < | <ns | < | < | <ns | < | 9 |
| 9 B3/9 B4 | < | < | > | <ns | ns | <ns | 7 |
| 9 F5/9 F6 | < | >ns | <ns | >ns | >ns | > | 7 |
| 14 B9/14 C1 | < | > | > | < | > | > | 8 |
| 14 D1/14 D2 | < | <ns | <ns | > | < | > | 7 |
| 37 i9/38 A1 | < | < | < | > | < | > | 7 |
| 38 A3/38 A4 | < | < | >ns | > | >ns | > | 7 |
| 38 F1/38 F2 | >ns | >ns | >ns | >ns | > | >ns | 6 |
| 42 B1/42 B2 | < | <ns | > | > | >ns | > | 6 |
| 43 C3/43 C4 | < | >ns | <ns | <ns | < | <ns | 6 |
| score | 1/9 | 4/6 | 5/5 | 6/4 | 5/5 | 7/3 | |

< = decreased in tumour relative to normal
> = increased in tumour relative to normal
ns = difference between normal and tumour not statistically significant
score = increased in tumour/decreased in tumour.

From these results it was demonstrated that all of the markers are expressed as RNA transcripts in both normal and tumour tissue. In general, fewer T1 transcripts are present in tumour tissue while Pspu43 transcripts are increased (significant association (P=0.0055) of raised Pspu43 in tumour vs normal tissue by 2×3 contingency table, Fisher's extact test). The loss of T1 is consistent with the findings for other cancers (Chang et al., 2001, Shields et al., 2002) and would correspond to a loss in cell cytoskeletal integrity and metastasis.

Pspu43 was upregulated in all tumours with Gleason grades between 6 and 8 relative to the normal sample taken from each individual where the difference between each sample was greater than the standard error. In the most severe lesion (14B9/C1, Gleason 9) there was relatively more Pspu43 marker in the normal portion of the prostate. It is questionable that this ‘normal’ sample reflected a normally functioning prostate given the extent of the disease in this particular organ (85% involvement of the gland) and this may reflect the advanced nature of the disease. It is quiet possible that the transcriptome characteristics of a tumour of Gleason Grade 9 are significantly different from less severe forms of the disease.

No overtly consistent pattern was seen in expression of the markers Pspu1, Pspu2, Pspu8 or T2. Therefore, though the Pspu markers 1, 2 and 8 were identified by our bioinformatics algorithm they did not prove able to differentiate tumour from normal prostate in this test.

### Conclusion

This experiment showed that Transgelin 1 and 2, and Pspu 1, 2, 8 and 43 are all expressed in prostate tissue regardless of disease state. No overtly cancer differentiating pattern of expression was demonstrated for markers T2, Pspu 1, Pspu2 or Pspu8. T1 however tended to be down-regulated in tumour tissue, consistent with the findings of others for lung, breast and colon cancers (Chang et al., 2001, Shields et al., 2002). Conversely, Pspu43 tended to be up-regulated in tumour tissue compared to the normal sample. This is significant. We know that this region of chromosome 8 is altered during the early disease process in many men. These results indicate that elevated Pspu43 is indicative of prostate cancer.

### REFERENCES

Altschul, S. F., Gish, W., Miller, W., Myers, E. W. & Lipman, D. J. (1990) Basic local alignment search tool. J. Mol. Biol. 215:403-410
Amundadottir L T, Sulem P, Gudmundsson J et al. (2006) A common variant associated with prostate cancer in European and African populations. Nat. Genet. (doi:10.1038/ng1808)
Beheshti B, Park P C, Sweet J M, Trachtenberg J, Jewett M A, Squire J A (2001) Evidence of chromosomal instability in prostate cancer determined by spectral karyotyping (SKY) and interphase fish analysis. Neoplasia 3: 62-69
Bello et al. (1997). Androgen responsive adult human prostatic epithelial cell lines immortalised by human papillomavirus 18. Carcinogenesis., 18, 1215-1223.
Bonaldo, Lennon & Soares (1996): Normalization and Subtraction: Two Approaches To Facilitate Gene Discovery. Genome Research 6, 791-806.
Bostwick D G (1996). Prospective origins of prostate carcinoma. Cancer 78: 330-336.
Chang J W, Jeon H B, Lee J H, Yoo J S, Chin J S, Kim J H, Yoo Y J (2001). Augmented expression of peroxidoxin I in lung cancer/Biochem Biophys Res Com/ 1289: 507-512
Cher M L, MacGrogan D, Bookstein R, Brown J A, Jenkins R B, Jensen R H (1994). Comparative genomic hybridization, allelic imbalance, and fluorescence in situ hybridization on chromosome 8 in prostate cancer. Genes, Chromosomes & Cancer 11: 153-162.
Fehm et al (2002). Cytogenetic evidence that circulating epithelial cells in Patients with Carcinomas are malignant. Clinical Cancer Research 8: 2073-2084.
Häggman M J, Wojno K J, Pearsall C P, Macoska J A (1997). Allelic loss of 8p sequences in prostatic intraepithelial neoplasia and carcinoma. Urology 50: 643-647.
Jefford C E, Irminger-Finger I (2006). Mechanisms of chromosome instability in cancers. Critical Reviews in Oncology/Hematology 59: 1-14
MacGrogan D, Levy A, Bostwick D, Wagner M, Wells D, Bookstein R, Loss of chromosome arm 8p loci in prostate cancer: Mapping by quantitative allelic imbalance (1994) Genes, Chromosomes & Cancer 10: 151-159.
Macoska J A, Trybus T M, Sakr W A et al., (1994). Fluoresence in situ hybridisation analysis of 8p allelic loss and chromosome 8 instability in human prostate cancer. Cancer Research 54: 5390-5395.
Macoska J A, Trybus T M, Benson P D et al., (1995). Evidence for three tumor suppressor gene loci on chromosome 8p in human prostate cancer. Cancer Research 55: 5390-5395.
Macoska J A, Trybus T M, Wojno K J (2000). 8p22 loss concurrent with 8c gain is associated with poor outcome in prostate cancer. Urology 55: 776-782.
Meng T C, Lee M S, Lin M F (2000) Interaction between protein tyrosine phosphatase and protein tyrosine kinase is involved in androgen-promoted growth of human prostate cancer cells. Oncogene 19:2664-77.
Peters L M, Anderson D W, Griffith A J, Grundfast K M, San Agustin T B, Madeo A C, Friedman T B, Morell R J (2002) Mutation of a transcription factor, TFCP2L3, causes progressive autosomal dominant hearing loss, DFNA28. Hum. Mol. Genet. 11: 2877-2885.
Rozen S, and Skaletsky H J (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386
Schuler, G. D., Boguski, M. S., Stewart, E. A., Stein, L. D., Gyapay, G., Rice, K., White, R. E., Rodriguez-Tome, P., Aggarwal, A., Bajorek, E., Bentolila, S., Birren, B. B., Butler, A., Castle, A. B., Chiannilkulchai, N., Chu, A., Clee, C., Cowles, S., Day, P. J., Dibling, T., Drouot, N., Dunham, I., Duprat, S., East, C., Hudson, T. J., et al. (1996) A gene map of the human genome, Science 274, 540-546.
Shields J M, Rogers-Graham K, Der C J (2002). Loss of transgelin in breast and colon tumours and in RIE-I cells by ras deregulation of gene expression through raf independent pathways. J Biol Chem 277, 9790-9799
Stajich, J. E., Block, D., Boulez, K., Brenner, S. E., Chervitz, S. A., Dagdigian, C., Fuellen, G., Gilbert, J. G., Korf, I., Lapp, H., Lehvaslaiho, H., Matsalla, C., Mungall, C. J., Osborne, B. I., Pocock, M. R., Schattner, P., Senger, M., Stein, L. D., Stupka, E., Wilkinson, M. D., Birney, E. (2002) The Bioperl toolkit: Perl modules for the life sciences, Genome Res. 12, 1611-1618.
Stanton, J L, Green D P L (2001). Meta-analysis of gene expression in mouse preimplantation embryo development. Molecular Human Reproduction, 7, 545-552. Webber M et al. (1997a). Acinar differentiation by non-malignant immortalized human prostatic epithelial cells and its loss by malignant cells. Carcinogenesis, 18, 1225-1231.
Webber M. et al. (1997). Immortalised and tumorogenic adult human prostatic epithelial cell lines: Characteristics and applications part 2. Tumorogenic cell lines. Prostate, 30, 58-64.

1-92. (canceled)

93. An isolated nucleic acid molecule, for use in a method of testing for, prognosing, diagnosing, or monitoring response to the treatment of, PIN or PRC in a patient, a molecule comprising the sequence of SEQ ID NO:3 or a functionally equivalent fragment or variant thereof, or a sequence which hybridises under stringent conditions to SEQ ID NO:3 or a fragment or variant thereof.

94. An isolated nucleic acid molecule of claim 93 which has 70%, 75%, 80%, 90%, 95%, or 99% sequence identity to SEQ ID NO:3.

95. An isolated nucleic acid molecule comprising an at least 10 nucleotide fragment of a nucleic acid sequence of claim 93, preferably SEQ ID NO:3, or a complement thereof, which fragment or complement hybridizes under stringent conditions to:
(a) a nucleic acid sequence of claim 93, preferably SEQ ID NO: 3 or a complement thereof;
(b) the full-length coding sequence of the cDNA corresponding to a nucleic acid sequence of claim 93 or a complement thereof;
(c) a reverse complement of (a) or (b).

96. The nucleic acid molecule of claim 93 which is at least 20, at least 30, at least 40, at least 50 nucleotides, at least 60 nucleotides, at least 70 nucleotides, at least 80 nucleotides, at least 90 nucleotides, or is at least 100 nucleotides in length.

97. A genetic construct which comprises a nucleic acid molecule of claim 93.

98. A genetic construct of claim 97 which is an expression construct.

99. A vector which comprises a genetic construct of claim 98.

100. A host cell which comprises a genetic construct or vector according to claim 97.

101. An isolated polypeptide encoded by a nucleic acid molecule of claim 93 or a functionally equivalent variant or fragment thereof.

102. An isolated polypeptide of claim 101 which is at least 5 amino acids in length.

103. An isolated polypeptide comprising a sequence of (a) SEQ ID NO:9, (b) SEQ ID: 10, (c) SEQ ID NO:11, (d) SEQ ID NO:12, (e) SEQ ID NO:13, or (f) SEQ ID NO;14; or a functionally equivalent variant or fragment of (a), (b), (c), (d), (e) or (f), or a polypeptide encoded by a sequence which hybridises under stringent conditions to a nucleic acid sequence encoding a polypeptide of any one of (a), (b), (c), (d), (e) or (f).

104. An isolated polypeptide of claim 101 wherein the polypeptide has at least: 70%, 75%, 80%, 85%, 90%, 95%, or 99% amino acid identity to a polypeptide of claim 101.

105. An antibody which specifically binds to a polypeptide of claim 101 or a functionally equivalent variant or fragment of the polypeptide.

106. An antibody according to claim 105 which is a polyclonal, monoclonal, single chain antibody or humanized antibody, or immunologically active fragment thereof.

107. An antibody according to claim 105 which is labelled with a detectable marker.

108. A method for recombinant production of a polypeptide according to claim 101, the method comprising the steps of:
(a) culturing a host cell comprising a genetic construct of claim 97, capable of expressing a polypeptide of claim 101; and
(b) selecting cells expressing the polypeptide of the invention;
(c) separating the expressed polypeptide from the cells; and optionally
(d) purifying the expressed polypeptide.

109. A method of claim 108 wherein the method comprises as a pre-step transfecting the host cells with the construct.

110. An array for use in a method of testing for, diagnosing, prognosing or monitoring the response to treatment of, PIN or PRC in a patient, the array comprising one or more nucleic acid sequences which bind PSPU43 (SEQ ID NO:3).

111. An array comprising one or more nucleic acid sequences of claim 93.

112. An array of claim 111 which further comprises one or more nucleic acid sequences which bind to one or more of transgelin 1 (SEQ ID NO:7), transgelin 2 (SEQ ID NO:8), PCA3 (SEQ ID NO:6), or prostate specific antigen (PSA) (SEQ ID NO:5).

113. An array as claimed in claim 110 wherein the nucleic acid sequences are RNA or DNA.

114. A method of screening for a compound that alters the expression of a nucleic acid molecule of claim 93, preferably PSPU43 (SEQ ID NO:3), the method comprising the steps of:
(a) contacting a cell that expresses the nucleic acid molecule with a test compound;
(b) determining the expression level of the nucleic acid molecule; and
(c) selecting the compound that alters the expression level compared to that level in the absence of the test compound.

115. A method of screening for a compound that alters the activity of a nucleic acid molecule of claim 93, preferably PSPU43 (SEQ ID NO:3), the method comprising:
(a) contacting a test compound with a peptide encoded by the nucleic acid molecule;
(b) detecting the biological activity of the peptide; and either:
(c) selecting the compound that alters the biological activity of the peptide in comparison with the biological activity detected in the absence of the compound; or
(d) selecting the compound that binds to the peptide.

116. A compound that alters expression or activity of a nucleic acid molecule of claim 93, preferably, PSPU43 (SEQ ID NO:3) selected by the screening method of claim 114.

117. A method for the treatment or prevention of Prostatic Intraepithelial Neoplasia (PIN) or Prostate Cancer (PRC), the method comprising administering a compound of 116.

118. A PIN or PRC expression profile, comprising a pattern of marker expression including a nucleic acid molecule of claim 93, preferably PSPU43 (SEQ ID NO:3).

119. A profile according to claim 118 which further comprises one or more markers selected from PCA3 (SEQ ID NO:6), transgelin 1 (SEQ ID NO:7), transgelin 2 (SEQ ID NO:8) and PSA (SEQ ID NO:5).

120. A method of treating or preventing PIN or PRC in a patient, the method comprising altering the expression level of a nucleic acid molecule of claim 93, preferably PSPU43(SEQ ID NO:3), in the patient, or by altering the activity of a polypeptide of claim 101.

121. A method of claim 120 wherein expression is inhibited by administering an antisense composition, siRNA composition, or ribozyme composition to the patient, the composition comprising one or more nucleotide sequences complementary to a nucleic acid molecule of claim 93.

122. A method of claim 121 wherein the composition is a vaccine.

123. A method of claim 120 wherein expression is inhibited by administering an antibody which specifically binds to a polypeptide of claim 101, preferably a polypeptide encoded by PSPU43 (SEQ ID NO:3).

124. A method of claim 123 wherein the antibody is a monoclonal antibody.

125. A method of treating or preventing PIN or PRC in a patient, the method comprising administering to said patient a compound that alters the expression or activity of a polypeptide of claim 101, preferably the polypeptide is encoded by SEQ ID NO:3.

126. A method of treating or preventing PIN or PRC in a patient wherein a nucleic acid molecule of claim 93, preferably is PSPU43 (SEQ ID NO:3), is over-expressed, the method comprising administering to said patient a compound that decreases the expression or activity of a polypeptide encoded by said nucleic acid molecule.

127. A composition comprising a pharmaceutically effective amount of a nucleic acid molecule according to claim 93, or a polypeptide according to claim 101 and a pharmaceutically acceptable carrier, diluent or excipient.

128. A composition comprising a pharmaceutically effective amount of an antisense-oligonucleotide, ribozyme or siRNA against a nucleic acid molecule of claim 93, preferably the nucleic acid molecule is PSPU43 (SEQ ID NO:3), and a pharmaceutically acceptable carrier, diluent or excipient.

129. A composition comprising a pharmaceutically effective amount of an antibody or fragment thereof that specifically binds to a polypeptide of claim 101, preferably the polypeptide is encoded by SEQ ID NO:3, and a pharmaceutically acceptable carrier, diluent or excipient.

130. A composition comprising a pharmaceutically effective amount of a compound selected by a screening method of claim 113 and a pharmaceutically acceptable carrier, diluent or excipient.

131. A method of treating or preventing PIN or PRC in a patient, the method comprising administering an effective amount of a compound of claim 116 to a patient in need thereof.

132. Use of PSPU43 (SEQ ID NO:3), or a polypeptide encoded by same in the preparation of a medicament for treating or preventing PIN or PRC in a patient.

133. Use of a nucleic acid molecule of claim 93, or a polypeptide of claim 101 in the preparation of a medicament for treating or preventing PIN or PRC in a patient.

134. An antisense-oligonucleotide, siRNA, or ribozyme against a nucleic acid molecule of claim 93, preferably against PSPU43 (SEQ ID NO:3).

135. An assay for use in a method of testing for, prognosing, diagnosing or monitoring response to the treatment of, PIN or PRC in a patient, the assay comprising detecting the presence of a nucleic acid molecule of claim 93, preferably PSPU43 (SEQ ID NO:3) in a sample, the method comprising:
(a) contacting the sample with a nucleotide probe which hybridises to a nucleic acid sequence of claim 93 under stringent hybridisation conditions; and
(b) detecting the presence of a hybridisation complex in the sample.

136. An assay of claim 135 wherein the probe is a labelled probe, preferably a fluorescently labelled probe.

137. An assay of claim 135 wherein the probe is a complement of SEQ ID NO:3.

138. A method of determining the level of expression of a nucleic acid molecule of claim 93, preferably PSPU43 (SEQ ID NO:3), in a patient sample, the method comprising direct or indirect measurement of the nucleic acid molecule.

139. A method of claim 138 wherein the nucleic acid molecule is employed in an in situ hybridisation or RT-PCR analysis.

140. A method of determining the level of expression of a nucleic acid molecule of claim 93, preferably PSPU43 (SEQ ID NO:3), in a patient sample, the method comprising:
(a) amplifying a DNA sequence of the nucleic acid molecule or complement thereof; or
(b) amplifying the cDNA sequence of the nucleic acid molecule or complement thereof; and
(c) measuring the level of one or more of DNA, cDNA or RNA in said sample.

141. A method of claim 140 wherein the DNA or cDNA is amplified using PCR.

142. A method of claim 138 wherein the level of DNA, cDNA, or RNA in the sample is measured using electrophoresis.

143. An assay for detecting the presence in a patient sample of a polypeptide of claim 101, the method comprising:
(a) contacting the sample with an antibody of claim 105; and
(b) detecting the presence of bound polypeptide in the sample.

144. The assay of claim 143 wherein said antibody is detectably labelled.

145. A method of diagnosing prostatic intraepithelial neoplasia (PIN), prostate cancer (PRC) or a predisposition to developing PIN or PRC in a patient, the method comprising determining the expression level of a nucleic acid molecule of any one of claim 93, preferably PSPU43 (SEQ ID NO:3), in a patient sample, wherein an alteration in expression level compared to a control level of said nucleic acid molecule indicates that the patient has PIN, PRC, or is at risk of developing PIN or PRC.

146. The method of claim 145 wherein the alteration is an increase in expression level.

147. The method according to claim 146 wherein the alteration in expression level is at least 10% above the normal control level.

148. The method of claim 144 wherein the control level is measured in a sample derived from normal prostate.

149. A method of testing for prostatic intraepithelial neoplasia (PIN), prostate cancer (PRC) or a predisposition to developing PIN or PRC status in a patient, the method comprising determining the expression level of a nucleic acid molecule of claim 93 in a patient sample, wherein an increase in expression level compared to a control level of said molecule indicates that the patient has PIN or PRC status, or is at risk of developing PIN or PRC.

150. A method of monitoring response to treatment of PIN or PRC in a patient, the method comprising determining the expression level of a nucleic acid molecule of claim 93, preferably PSPU43 (SEQ ID NO:3), in a patient sample, and comparing the level of said nucleic acid molecule to a control level, wherein a statistically significant change in the determined level from the control level is indicative of a response to the treatment.

151. A method as claimed in claim 145 which further comprises determining the level of one or more additional markers of PIN or PRC and comparing the levels to marker levels from a control, wherein a significant deviation in the levels from a control level, together with a statistically significant increase in the level of a nucleic acid molecule of claim 93, preferably PSPU43 (SEQ ID NO:3) is indicative of PRC or PIN, or can be used to monitor PIN or PRC status.

152. A method of claim 151 wherein the additional markers are selected from the group consisting of transgelin 1 (SEQ ID NO:7), prostate specific antigen (SEQ ID NO:5), and PCA3 (SEQ ID NO:6).

153. A method of claim 145 wherein the sample is a urine, lymph, blood, plasma, semen, prostate massage fluid, or prostate tissue sample.

154. A method of claim 145 wherein transgelin 2 (SEQ ID NO: 8) is used as a reference marker.

155. Use of transgelin 2 (SEQ ID NO: 8) as a reference marker in a method of claim 145.

156. Use of a nucleic acid molecule of claim 93, preferably PSPU43 (SEQ ID NO:3), in a method of testing for, diagnosing, prognosing or monitoring response to the treatment of, PIN or PRC in a patient.

157. A kit for detecting the presence of a nucleic acid molecule of claim 93, preferably PSPU43 (SEQ ID NO:3), in a sample, the kit comprising at least one container comprising the nucleic acid molecule of claim 93, and one or more reagents for detecting said nucleic acid molecule.

158. A kit comprising one or more detection reagents which bind to a nucleic acid molecule of claim 93, preferably PSPU43 (SEQ ID NO: 3), or a polypeptide encoded by said nucleic acid molecule.

159. A kit as claimed in claim 157 further comprising one or more of:
(a) a nucleic acid molecule encoding transgelin 1 (SEQ ID NO:7) or a complement thereof;
(b) a nucleic acid molecule encoding transgelin 2 (SEQ ID NO:8) or a complement thereof;
(c) a nucleic acid molecule encoding PCA3 (SEQ ID NO:6) or a complement thereof;
(d) a nucleic acid molecule encoding PSA (SEQ ID NO:5) or a complement thereof; and
(e) all of (a) to (d).

160. A non-human animal, preferably a mouse, having a genome wherein a nucleic acid molecule of claim 93, preferably PSPU43 (SEQ ID NO:3) is altered, disrupted, eliminated or added.


SEQUENCE LISTING (88 sequences)

SEQ ID NO: 1 - DNA, 368 bp, Homo sapien
aagaattcggcacgaggaatatttaagggtaaaatttttctacttttaaagcttaaaaaaatgtttttttactactgtaaaagtaatgcagagaaatgttcacttaccaaacacatacctttgtaaaaatcaccacttaaagtttgtttctaaagattttaggacaccaagatgcaaataatatttttggctgttacctgctctttcactactgctgagtctgcagtggcaagatagctacacagtacctcagccctcctgctcagtttttaacatctattgataatactaattacaagaaaatttaaaatgtctttttgcaaaaaaataccataagcagtcaaaacacaattaaaaaaaaaaaaaaa

SEQ ID NO: 2 - DNA, 394 bp, Homo sapien
  misc_feature 143: n = A or absent
gcaaaattaggacattcccagataaacaaataaagagacttcatcactaataaacatgccctgtatgaaatactaagaggagtccttggggcggacatgaaaggacactagacaaatccacatgaagacgtaaagattgccangtgaagtaactacataggcaaatataaacgatagtataaatgtatttttgtttgtgactcttttttttgtgctgatttaaaagacagcttcatagaaaaaaattataaatctgtgttgatgggcacacagtgcacaaagatgtaatattaatacaataacagcacaggaatggagagggaacagaggaatacaaaatctaactagacttataacaagcaaatggattgatttaattaaaaaaaaaaaaaaa

SEQ ID NO: 3 - DNA, 392 bp, Homo sapien
tttattaacaaatataaagtaccagacactccaagtgcttagaaaattaaattaactcatttaattttactaaaaacctgtgagataagggactattgttatgcccatttttcagatgaggaaagtgggacagagtttactgagtggtagagatgagatttgaacccaggcaagcaggctctaggtctggactcttggtcactctgctctgtccccacactaagccaggtatctgggcttgtgtacatagtcaggtaagtggaaatcacaggaattgctagaggactccagtagagggatgattttcatgaatgttctttcatctcaagaaagcaaaggctgctttttgggtgcaaaggagtttggctagaaggaagatctggagatagagc

SEQ ID NO: 4 - DNA, 1320 bp, Homo sapien
  misc_feature 931: s can be C or G
cacgaggcgagaaacacccaagaatgatcaattaaaaaaaaaaaagaaagaaagaaagaaagaaaagaagcagagttggactcagcgggaaaataggcgtgctaccacctcaggagagttccagggaagaaccccacccgcactccaatgaggtcacaatggctggagctctgaggggcccaggctccctgagccaggaggagaggagaaagtccaaggaaagatggctggcagtcacccctacttcaacctgcctgactccacacacccatcgccgccctccgctccacccagcctccgctggcaccagcgctgccagccctctggtgccaccaatggcctgctggtggccctgctgggtgggggcctgcctgctggcttcgtgggccccctttctcgtatggcttaccagggttccaacctgccctcgctggagctgctcatctgtcgatgcctcttccacctccctattgccctgctacttaaactgcgtggcgacccccttctgggacctcctgacatccgaggctgggcctgcttctgtgccctgctcaacgtcctcagcattggatgtgcctacagtgcagttcaggtggtgcccgctggcaacgctgccactgttcgcaaaggttcttccaccgtatgctccgctgtcctcaccctactgccttgagagccagggtctcggtggctacgagtggtgtggactgttgggcagcatcctaggactaatcatcattctgggacctggactctggacactaccaggaggggaccacaggtgtctacaccaccctgggctatgtgcaggctttcctgggaggcctggcgctgtccctggggctaatctggtctatcgtatctctgcactttcccatcctgcctcccaacagtggccttcctatctggcttggatggggctgctgggctstgtgccaggcctctttgtgctgcagacccccgtgttgcccagtgacctcctgagttggagttgtgtggggggcagaggggatcctcgccswtkggtctcccttcacatgggtgtgggctatgcgggtcaccaaggccccacaccctgccctggtgtgcgctgtcctgcattccgaggtggttgtggcccttatactgcagtattatatgctccatgagactgtggcactttctgacatcatgggggcaggggttgtgctgggcagcattgccatcattacagcccggaacctcagctgtgagaggacagggaaggtggaggagtgagatagaacttgggagcccgggggttgggagggacagggataaataaagacaaagactgaagac

SEQ ID NO: 5 - DNA, 1906 bp, Homo sapien
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccccaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 6 - DNA, 3922 bp, Homo sapien
acagaagaaatagcaagtgccgagaagctggcatcagaaaaacagaggggagatttgtgtggctgcagccgagggagaccaggaagatctgcatggtgggaaggacctgatgatacagaggaattacaacacatatacttagtgtttcaatgaacaccaagataaataagtgaagagctagtccgctgtgagtctcctcagtgacacagggctggatcaccatcgacggcactttctgagtactcagtgcagcaaagaaagactacagacatctcaatggcaggggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcatttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagcacagcctctccccatccctccagccttatctgtcatcaccatcaacccctcccataccacctaaacaaaatctaacttgtaattccttgaacatgtcaggacatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgtgagtgtacatgccaaagtgtgcctctctctcttgacccattatttcagacttaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 7 - DNA, 1574 bp, Homo sapien
tcaccacggcggcagccctttaaacccctcacccagccagcgccccatcctgtctgtccgaacccagacacaagtcttcactccttcctgcgagccctgaggaagccttgtgagtgcattggctggggcttggagggaagttgggctggagctggacaggagcagtgggtgcatttcaggcaggctctcctgaggtcccaggcgccagctccagctccctggctagggaaacccaccctctcagtcagcatgggggcccaagctccaggcagggtgggctggatcactagcgtcctggatctctctcagactgggcagccccgggctcattgaaatgccccggatgacttggctagtgcagaggaattgatggaaaccaccggggtgagagggaggctccccatctcagccagccacatccacaaggtgtgtgtaagggtgcaggcgccggccggttaggccaaggctctactgtctgttgcccctccaggagaacttccaaggagctttccccagacatggccaacaagggtccttcctatggcatgagccgcgaagtgcagtccaaaatcgagaagaagtatgacgaggagctggaggagcggctggtggagtggatcatagtgcagtgtggccctgatgtgggccgcccagaccgtgggcgcttgggcttccaggtctggctgaagaatggcgtgattctgagcaagctggtgaacagcctgtaccctgatggctccaagccggtgaaggtgcccgagaacccaccctccatggtcttcaagcagatggagcaggtggctcagttcctgaaggcggctgaggactatggggtcatcaagactgacatgttccagactgttgacctctttgaaggcaaagacatggcagcagtgcagaggaccctgatggctttgggcagcttggcagtgaccaagaatgatgggcactaccgtggagatcccaactggtttatgaagaaagcgcaggagcataagagggaattcacagagagccagctgcaggagggaaagcatgtcattggccttcagatgggcagcaacagaggggcctcccaggccggcatgacaggctacggacgacctcggcagatcatcagttagagcggagagggctagccctgagcccggccctcccccagctccttggctgcagccatcccgcttagcctgcctcacccacacccgtgtggtaccttcagccctggccaagctttgaggctctgtcactgagcaatggtaactgcacctgggcagctcctccctgtgcccccagcctcagcccaacttcttacccgaaagcatcactgccttggcccctccctcccggctgcccccatcacctctactgtctcctccctgggctaagcaggggagaagcgggctgggggtagcctggatgtgggccaagtccactgtcctccttggcggcaaaagcccattgaagaagaaccagcccagcctgccccctatcttgtcctggaatatttttggggttggaactcaaaaaaaaaaaaaaaaaatcaatcttttctcaaaaaaaaaaaaaaaaaa

SEQ ID NO: 8 - DNA, 1360 bp, Homo sapien
gcccttgccttgagtcagtgcgctgctctccagcccgcttgaacgctccccgcagccaccgccacccattggaatggccaacaggggacctgcatatggcctgagccgggaggtgcagcagaagattgagaaacaatatgatgcagatctggagcagatcctgatccagtggatcaccacccagtgccgaaaggatgtgggccggccccagcctggacgcgagaacttccagaactggctcaaggatggcacggtgctatgtgagctcattaatgcactgtaccccgaggggcaggccccagtaaagaagatccaggcctccaccatggccttcaagcagatggagcagatctctcagttcctgcaagcagctgagcgctatggcattaacaccactgacatcttccaaactgtggacctctgggaaggaaagaacatggcctgtgtgcagcggacgctgatgaatctgggtgggctggcagtagcccgagatgatgggctcttctctggggatcccaactggttccctaagaaatccaaggagaatcctcggaacttctcagataaccagctgcaagagggcaagaacgtgatcgggttacagatgggcaccaaccgcggggcgtctcaggcaggcatgactggctacgggatgccacgccagatcctctgatcccaccccaggccttgcccctgccctcccacgaatggttaatatatatgtagatatatattttagcagtgacattcccagagagccccagagctctcaagctcctttctgtcagggtggggggttcagcctgtcctgtcacctctgaggtgcctgctggcatcctctcccccatgcttactaatacattcccttccccatagccatcaaaactggaccaactggcctcttcctttcccctgggaccaaaatttaggggcctcagtccctcaccgccatgccctggcctattctgtctctccttcttccccctggcctgttctgtctctgagctctgtgtcctccgttcattccatggctgggagtcactgatgctgcctctgccttctgatgctggactggccttgcttctacaagtatgcttctcccacagctgtggctgcaggaacttaatttatagggaggagcctgtggcagctgctgccccagccacagctgcactgactgtgctcaccacacatctggggcagccttccctggcaggggccctcgtggcttctcattttccattcccttcactgtggctaaggggtggggtgaggggatggagagggagggctgcctaccatggtctggggcttgaggaagatgagtttgttgatttaaataaagaatttgtcatttttg

SEQ ID NO: 9 - protein, 13 aa, Homo sapien
FINKYKVPDTPSA

SEQ ID NO: 10 - protein, 17 aa, Homo sapien
LLTNIKYQTLQVLRKLN

SEQ ID NO: 11 - protein, 10 aa, Homo sapien
STRHSKCLEN

SEQ ID NO: 12 - protein, 28 aa, Homo sapien
ALSPDLPSSQTPLHPKSSLCFLEMKEHS

SEQ ID NO: 13 - protein, 23 aa, Homo sapien
LYLQIFLLAKLLCTQKAAFAFLR

SEQ ID NO: 14 - protein, 7 aa, Homo sapien
SISRSSF

SEQ ID NO: 15 - DNA, 29 bp, Artificial Sequence
  A synthetic primer
attgtgttttgactgcttatggtattttt

SEQ ID NO: 16 - DNA, 28 bp, Artificial Sequence
  A synthetic primer
cagagaaatgttcacttaccaaacacat

SEQ ID NO: 17 - DNA, 30 bp, Artificial Sequence
  A synthetic primer
aatcaatccatttgcttgttataagtctag

SEQ ID NO: 18 - DNA, 29 bp, Artificial Sequence
  A synthetic primer
aaattaggacattcccagataaacaaata

SEQ ID NO: 19 - DNA, 21 bp, Artificial Sequence
  A synthetic primer
aaccgtgagaagatgacccag

SEQ ID NO: 20 - DNA, 21 bp, Artificial Sequence
  A synthetic primer
gtgaggatcttcatgaggtag

SEQ ID NO: 21 - DNA, 19 bp, Artificial Sequence
  A synthetic primer
ccctttctcgtatggctta

SEQ ID NO: 22 - DNA, 20 bp, Artificial Sequence
  A synthetic primer
gctgtgagaggacagggaag

SEQ ID NO: 23 - DNA, 27 bp, Artificial Sequence
  A synthetic primer
aacaaatataaagtaccagacactcca

SEQ ID NO: 24 - DNA, 23 bp, Artificial Sequence
  A synthetic primer
atctccagatcttccttctagcc

SEQ ID NO: 25 - protein, 238 aa, Homo sapien
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSWVILITELTMPALPMVLHGSLVPWRGGV

SEQ ID NO: 26 - protein, 201 aa, Homo sapien
MANKGPSYGMSREVQSKIEKKYDEELEERLVEWIIVQCGPDVGRPDRGRLGFQVWLKNGVILSKLVNSLYPDGSKPVKVPENPPSMVFKQMEQVAQFLKAAEDYGVIKTDMFQTVDLFEGKDMAAVQRTLMALGSLAVTKNDGHYRGDPNWFMKKAQEHKREFTESQLQEGKHVIGLQMGSNRGASQAGMTGYGRPRQIIS

SEQ ID NO: 27 - protein, 199 aa, Homo sapien
MANRGPAYGLSREVQQKIEKQYDADLEQILIQWITTQCRKDVGRPQPGRENFQNWLKDGTVLCELINALYPEGQAPVKKIQASTMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 28 - DNA, 329 bp, Homo sapien
ctttctttttttttgctctatctccagatcttccttctagccaaactcctttgcacccaaaaagcagcctttgctttcttgagatgaaagaacattcatgaaaatcatccctctactggagtcctctagcaattcctgtgatttccacttacctgactatgtacacaagcccagatacctggcttagtgtggggacagagcagagtgaccaagagtccagacctagagcctgcttgcctgggttcaaatctcatctctaccactcagtaaactctgtcccactttcctcatctgaaaaatgggcataacaatagtcccttatctacagg

SEQ ID NO: 29 - DNA, 312 bp, Homo sapien
ttttttttttttttgctctatctccagatcttccttctagccaaactcctttgcacccaaaaagcagcctttgctttcttgagatgaaagaacattcatgaaaatcatccctctactggagtcctctagcaattcctgtgatttccacttacctgactatgtacacaagcccagatacctggcttagtgtggggacagagcagagtgaccaagagtccagacctagagcctgcttgcctgggttcaaatctcatctctaccactcagtaaactctgtcccactttcctcatctgaaaaatgggcataacaat

SEQ ID NO: 30 - DNA, 329 bp, Homo sapien
tttttttttttttttgctctatctccagatcttccttctagccaaactcctttgcacccaaaaagcagcctttgctttcttgagatgaaagaacattcatgaaaatcatccctctactggagtcctctagcaattcctgtgatttccacttacctgactatgtacacaagcccagatacctggcttagtgtggggacagagcaaagtgaccaagagtccaaacctagagcctgcttgcctgggttcaaatctcatctctaccactcagtaaactctgtcccactttcctcatctgaaaaatgggcataacaatagtcccttatctcaca

SEQ ID NO: 31 - DNA, 373 bp, Homo sapien
ctttctttttttttgctctatctccagatcttccttctagccaaactcctttgcacccaaaaagcagcctttgctttcttgagatgaaagaacattcatgaaaatcatccctctactggagtcctctagcaattcctgtgatttccacttacctgactatgtacacaagcccagatacctggcttagtgtggggacagagcagagtgaccaagagtccagacctagagcctgcttgcctgggttcaaatctcatctctaccactcagtaaactctgtcccactttcctcatctgaaaaatgggcataacaatagtcccttatctcacaggtttttagtaaaattaaatgagttaatttaattttctaagcact

SEQ ID NO: 32 - DNA, 421 bp, Homo sapien
tttattaacaaatataaagtaccagacactccaagtgcttagaaaattaaattaactcatttaattttactaaaaacctgtgagataagggactattgttatgcccatttttcagatgaggaaagtgggacagagtttactgagtggtagagatgagatttgaacccaggcaagcaggctctaggtctggactcttggtcactctgctctgtccccacactaagccaggtatctgggcttgtgtacatagtcaggtaagtggaaatcacaggaattgctagaggactccagtagagggatgattttcatgaatgttctttcatctcaagaaagcaaaggctgctttttgggtgcaaaggagtttggctagaaggaagatctggagatagagcaaaaaaaaagaaagaaaaaaaaaaaaaaa

SEQ ID NO: 33 - DNA, 296 bp, Homo sapien
ttttttttttgctctatctccagatcttccttctagccaaactcctttgcacccaaaaagcagcctttgctttcttgagatgaaagaacattcatggaaatcatccctctactggagtcctctagcaattcctgtgatttccacttacctgactatgtacacaagcccagatacctggcttagtgtggggacagagcagagtgaccaagagtccagacctagagcctgcttgcctgggttcaaatctcatctctaccactcagtaaactctgtcccactttcctcatctggtcgac

SEQ ID NO: 34 - protein, 51 aa, Homo sapien
MFLHISSPFKYPHTQEAQKEAQRSLGEMPGRHLGSSMSLALCLVPLVRQGH

SEQ ID NO: 35 - protein, 51 aa, Homo sapien
MFLHISSPFKYPHTQEAQKEAQRSLGEMPGRHLGSSMSLALCLVPLVREGH

SEQ ID NO: 36 - protein, 35 aa, Homo sapien
KIKLTHLILHKTCEIRDYCYAHFSDEESGTEFTEW

SEQ ID NO: 37 - protein, 33 aa, Homo sapien
DLNPGKQALGLDSWSLCCVPTLSQVSGLVYIVR

SEQ ID NO: 38 - protein, 11 aa, Homo sapien
VECTGIARGLQ

SEQ ID NO: 39 - protein, 32 aa, Homo sapien
RDDFHECSFISRKQRLLFGCKGVWLEGRSGDR

SEQ ID NO: 40 - protein, 4 aa, Homo sapien
KPVR

SEQ ID NO: 41 - protein, 24 aa, Homo sapien
GTIVMPIFQMRKVGQSLLSGRDEI

SEQ ID NO: 42 - protein, 6 aa, Homo sapien
TQASRL

SEQ ID NO: 43 - protein, 12 aa, Homo sapien
VWTLGHSALSPH

SEQ ID NO: 44 - protein, 8 aa, Homo sapien
ARYLGLCT

SEQ ID NO: 45 - protein, 39 aa, Homo sapien
SGKWKSQELLEDSSRGMIFMNVLSSQESKGCFLGAKEFG

SEQ ID NO: 46 - protein, 7 aa, Homo sapien
KEDLEIE

SEQ ID NO: 47 - protein, 10 aa, Homo sapien
INSFNFTKNL

SEQ ID NO: 48 - protein, 11 aa, Homo sapien
DKGLLLCPFFR

SEQ ID NO: 49 - protein, 7 aa, Homo sapien
GKWDRVY

SEQ ID NO: 50 - protein, 45 aa, Homo sapien
VVEMRFEPRQAGSRSGLLVTLLCPHTKPGIWACVHSQVSGNHRNC

SEQ ID NO: 51 - protein, 6 aa, Homo sapien
RTPVEG

SEQ ID NO: 52 - protein, 25 aa, Homo sapien
MFFHLKKAKAAFWVQRSLARRKIWR

SEQ ID NO: 53 - protein, 68 aa, Homo sapien
KSSLYWSPLAIPVISTYLTMYTSPDTWLSVGTEQSDQESRPRACLPGFKSHLYHSVNSVPLSSSEKWA

SEQ ID NO: 54 - protein, 11 aa, Homo sapien
SLISQVFSKIK

SEQ ID NO: 55 - protein, 5 aa, Homo sapien
VNLIF

SEQ ID NO: 56 - protein, 12 aa, Homo sapien
ALGVSGTLYLLI

SEQ ID NO: 57 - protein, 13 aa, Homo sapien
KNIHENHPSTGVL

SEQ ID NO: 58 - protein, 4 aa, Homo sapien
FPLT

SEQ ID NO: 59 - protein, 37 aa, Homo sapien
LCTQAQIPGLVWGQSRVTKSPDLEPACLGSNLISTTQ

SEQ ID NO: 60 - protein, 29 aa, Homo sapien
TLSHFPHLKNGHNNSPLSHRFLVKLNELI

SEQ ID NO: 61 - protein, 13 aa, Homo sapien
FSKHLECLVLYIC

SEQ ID NO: 62 - protein, 13 aa, Homo sapien
PNSFAPKKQPLLS

SEQ ID NO: 63 - protein, 34 aa, Homo sapien
DERTFMKIIPLLESSSNSCDFHLPDYVHKPRYLA

SEQ ID NO: 64 - protein, 6 aa, Homo sapien
CGDRAE

SEQ ID NO: 65 - protein, 5 aa, Homo sapien
PRVQT

SEQ ID NO: 66 - protein, 22 aa, Homo sapien
SLLAWVQISSLPLSKLCPTFLI

SEQ ID NO: 67 - protein, 13 aa, Homo sapien
KMGITIVPYLTGF

SEQ ID NO: 68 - protein, 17 aa, Homo sapien
FNFLSTWSVWYFIFVNK

SEQ ID NO: 69 - DNA, 20 bp, Artificial Sequence
  A synthetic primer
cttccagaactggctcaagg

SEQ ID NO: 70 - DNA, 20 bp, Artificial Sequence
  A synthetic primer
gagaagagcccatcatctcg

SEQ ID NO: 71 - DNA, 23 bp, Artificial Sequence
  A synthetic primer
cactgcatcaggaacaaaagcgt

SEQ ID NO: 72 - DNA, 21 bp, Artificial Sequence
  A synthetic primer
catcacctggcctgaggaatc

SEQ ID NO: 73 - DNA, 23 bp, Artificial Sequence
  A synthetic primer
ggctctaggtctggactcttggt

SEQ ID NO: 74 - DNA, 25 bp, Artificial Sequence
  A fluorogenic probe
tgctctgtccccacactaagccagg

SEQ ID NO: 75 - DNA, 25 bp, Artificial Sequence
  A synthetic primer
cctgactatgtacacaagcccagat

SEQ ID NO: 76 - DNA, 18 bp, Artificial Sequence
  A synthetic primer
ggctgggcctgcttctgt

SEQ ID NO: 77 - DNA, 26 bp, Artificial Sequence
  A flourescent probe
ctcaacgtcctcagcattggatgtgc

SEQ ID NO: 78 - DNA, 18 bp, Artificial Sequence
  A synthetic primer
gcggagcatacggtggaa

SEQ ID NO: 79 - DNA, 29 bp, Artificial Sequence
  A synthetic primer
ccctgtatgaaatactaagaggagtcctt

SEQ ID NO: 80 - DNA, 31 bp, Artificial Sequence
  A flourescent probe
cggacatgaaaggacactagacaaatccaca

SEQ ID NO: 81 - DNA, 34 bp, Artificial Sequence
  A synthetic primer
ctatcgtttatatttgcctatgtagttacttcac

SEQ ID NO: 82 - DNA, 22 bp, Artificial Sequence
  A synthetic primer
tggctgttacctgctctttcac

SEQ ID NO: 83 - DNA, 29 bp, Artificial Sequence
  A flourescent probe
agctatcttgccactgcagactcagcagt

SEQ ID NO: 84 - DNA, 22 bp, Artificial Sequence
  A synthetic primer
caggagggctgaggtactgtgt

SEQ ID NO: 85 - DNA, 20 bp, Artificial Sequence
  A synthetic primer
aagaatgatgggcactaccg

SEQ ID NO: 86 - DNA, 20 bp, Artificial Sequence
  A synthetic primer
actgatgatctgccgaggtc

SEQ ID NO: 87 - DNA, 20 bp, Artificial Sequence
  A synthetic primer
cttccagaactggctcaagg

SEQ ID NO: 88 - DNA, 20 bp, Artificial Sequence
  A synthetic primer
gagaagagcccatcatctcg